(19) United States
(12) Patent Application Publication
(10) Pub. No.: US 2023/0039268 A1
(43) Pub. Date: Feb. 9, 2023

(54) ANTI-PD-L1 ANTIBODY FORMULATIONS

(71) Applicant: Genentech, Inc., South San Francisco, CA (US)

(72) Inventors: Ada HUI, South San Francisco, CA (US); Judith ZHU-SHIMONI, South San Francisco, CA (US)

(73) Assignee: Genentech, Inc., South San Francisco, CA (US)

(21) Appl. No.: 17/783,229

(22) PCT Filed: Dec. 7, 2020
(86) PCT No.: PCT/US2020/063620

§ 371 Date: Jun. 7, 2022

Related U.S. Application Data
(60) Provisional application No. 62/945,730, filed on Dec. 9, 2019.

(51) Int. Cl. A61K 39/395; C07K 16/28; A61K 47/22; A61K 47/26; A61K 47/20; A61K 47/42

(52) U.S. Cl. CPC A61K 39/39591; C07K 16/2827; A61K 47/22; A61K 47/26; A61K 47/20; A61K 47/42; C07K 2317/24; A61K 2039/54

(57) ABSTRACT

The invention provides liquid pharmaceutical formulations comprising an anti-PD-L1 antibody, such as liquid pharmaceutical formulations for subcutaneous administration. The invention also provides methods for making such formulations and methods of using such formulations.

### CROSS REFERENCE TO RELATED APPLICATION

This application claims priority benefit of U.S. Provisional Application No. 62/945,730 filed Dec. 9, 2019, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 146392049940SEQLIST.TXT, date recorded: Nov. 22, 2020, size: 9 KB).

### TECHNICAL FIELD

The invention provides liquid pharmaceutical formulations comprising an anti-PD-L1 antibody, such as liquid pharmaceutical formulations for subcutaneous administration. The invention also provides methods for making such formulations and methods of using such formulations.

### BACKGROUND OF THE INVENTION

The pharmaceutical use of antibodies has increased over the past years. In many instances such antibodies are either injected or infused via the intravenous (IV) route. Unfortunately the amount of antibody that can be administered via the intravenous route is limited by the physico-chemical properties of the antibody, in particularly by its solubility and stability in a suitable liquid formulation and by the volume of the infusion fluid. Alternative administration pathways are subcutaneous or intramuscular injection. These injection pathways require high protein concentration in the final solution to be injected (Shire, S. J., Shahrokh, Z. et al, “Challenges in the development of high protein concentration formulations”, J. Pharm. Sci. 2004; 93(6): 1390-1402; Roskos, L. K., Davis C. G. et al, “The clinical pharmacology of therapeutic antibodies”, Drug Development Research 2004; 61(3): 108-120). In order to increase the volume, and thereby the therapeutic dose, it has been proposed to use glycosaminoglycanase enzyme(s) in order to increase the interstitial space into which the antibody formulation can be injected (WO2006/091871).

There is a desire to provide highly concentrated, stable pharmaceutical formulations of therapeutically active antibodies for subcutaneous injection. The advantage of subcutaneous injections is that it allows the medical practitioner to perform it in a rather short intervention with the patient. Moreover the patient can be trained to perform the subcutaneous injection by himself. Usually injections via the subcutaneous route are limited to approximately 2 ml. For patients requiring multiple doses, several unit dose formulations can be injected at multiple sites of the body surface. No highly concentrated, stable pharmaceutical anti-PD-L1 antibody formulation suitable for subcutaneous administration is currently available on the market. There is therefore a desire to provide such highly concentrated, stable pharmaceutical formulations of therapeutically active antibodies for subcutaneous injection. The injection of parenteral drugs into the hypodermis is generally limited to volumes of less than 2 ml due to this viscoelastic resistance to hydraulic conductance in the subcutaneous (SC) tissue and generated backpressure upon injection (Aukland K. and Reed R., “Interstitial-Lymphatic Mechanisms in the control of Extracellular Fluid Volume”, Physiology Reviews“, 1993; 73: 1-78) as well as due to the perceptions of pain.

The preparation of high concentration protein formulations is very challenging and there is a need to adapt each formulation to the particular proteins used because each protein has a different aggregation behavior. Aggregates are suspected to cause immunogenicity of therapeutic proteins in at least some of the cases. Immunogenic reaction against protein or antibody aggregates may lead to neutralizing antibodies which may render the therapeutic protein or antibody ineffective. It appears that the immunogenicity of protein aggregates is most problematic in connection with subcutaneous injections, whereby repeated administration increases the risk of an immune response.

PD-L1 is overexpressed in many cancers and is often associated with poor prognosis (Okazaki T et al., Intern. Iramim. 2007 19(7):813) (Thompson R H et al., Cancer Res 2006, 66(7):3381). Interestingly, the majority of tumor infiltrating T lymphocytes predominantly express PD-1, in contrast to T lymphocytes in normal tissues and peripheral blood T lymphocytes indicating that up-regulation of PD-1 on tumor-reactive T cells can contribute to impaired antitumor immune responses (Blood 2009 114(8): 1537). This may be due to exploitation of PD-L1 signaling mediated by PD-L1 expressing tumor cells interacting with PD-1 expressing T cells to result in attenuation of T cell activation and evasion of immune surveillance (Sharpe et al., Nat Rev 2002) (Keir M E et al, 2008 Annu. Rev. Immunol. 26:677). Therefore, inhibition of the PD-L1/PD-1 interaction may enhance CD8+ T cell-mediated killing of tumors,

Therapeutic targeting PD-1 and other molecules which signal through interactions with PD-1, such as programmed death ligand 1 (PD-L1) and programmed death ligand 2 (PD-L2) are an area of intense interest. The inhibition of PD-L1 signaling has been proposed as a means to enhance T cell immunity for the treatment of cancer and infection, including both acute and chronic (e.g., persistent) infection. Formulations of anti-PD-L1 antibodies that can be used for intravenous infusion have been described (see US 2016/0319022). However, as an optimal formulation for an anti-PD-L1 antibody that is suitable for subcutaneous injection has yet to be developed, a significant unmet medical need exists.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

In one aspect, provided herein is a liquid pharmaceutical formulation, the formulation comprising a monoclonal anti-PD-L1 antibody in a concentration of about 100 g/L to about 150 g/L, histidine acetate in a concentration of about 15 mM to about 25 mM, sucrose in a concentration of about 200 mM to about 280 mM, polysorbate in a concentration of about 0.04% (w/v) to about 0.08% (w/v), methionine in a concentration of about 5 mM to about 15 mM, and pH of about 5.6 to about 6.0, wherein the monoclonal antibody comprises

(a) a light chain variable region comprising:
(1) HVR-L1 comprising the amino acid sequence RASQDVSTAVA (SEQ ID NO: 1);
(2) HVR-L2 comprising the amino acid sequence SASFLYS (SEQ ID NO:2);
(3) HVR-L3 comprising the amino acid sequence QQYLYHPAT (SEQ ID NO:3); and

(b) a heavy chain variable region comprising:
(1) HVR-H1 comprising the amino acid sequence GFTFSDSWIH (SEQ ID NO:4);
(2) HVR-H2 comprising the amino acid sequence AWISPYGGSTYYADSVKG (SEQ ID NO:5);
(3) HVR-H3 comprising the amino acid sequence WPGGFDY (SEQ ID NO:6). In some embodiments, the monoclonal antibody in the formulation is in a concentration of about 120 g/L to about 130 g/L. In some embodiments, the monoclonal antibody in the formulation is in a concentration of about 125 g/L. In some embodiments, the histidine acetate is in a concentration of about 17 mM to about 22 mM. In some embodiments, the histidine acetate is in a concentration of about 20 mM. In some embodiments, the sucrose is in a concentration of about 220 mM to about 260 mM. In some embodiments, the sucrose is in a concentration of about 240 mM. In some embodiments, the pH is about 5.8. In some embodiments, the polysorbate in the formulation is polysorbate 20. In some embodiments, the polysorbate is in a concentration of about 0.05% (w/v) to about 0.07% (w/v). In some embodiments, the polysorbate is in a concentration of about 0.06% (w/v). In some embodiments, the methionine is in a concentration of about 10 mM. In some embodiments, the formulation further comprises a hyaluronidase enzyme. In some embodiments, the hyaluronidase enzyme is recombinant human hyaluronidase (rHuPH20). In some embodiments, the hyaluronidase enzyme is in a concentration of about 1000 U/ml to about 3000 U/ml. In some embodiments, the hyaluronidase enzyme is in a concentration of about 2000 U/ml.

In one aspect, provided herein is a liquid pharmaceutical formulation, the formulation comprising a monoclonal anti-PD-L1 antibody in a concentration of about 100 g/L to about 150 g/L, histidine acetate in a concentration of about 15 mM to about 25 mM, sucrose in a concentration of about 200 mM to about 280 mM, polysorbate in a concentration of about 0.01% (w/v) to about 0.03% (w/v), and pH of about 5.3 to about 5.7, wherein the monoclonal antibody comprises

(a) a light chain variable region comprising:
(1) HVR-L1 comprising the amino acid sequence RASQDVSTAVA (SEQ ID NO: 1);
(2) HVR-L2 comprising the amino acid sequence SASFLYS (SEQ ID NO:2);
(3) HVR-L3 comprising the amino acid sequence QQYLYHPAT (SEQ ID NO:3); and

(b) a heavy chain variable region comprising:
(1) HVR-H1 comprising the amino acid sequence GFTFSDSWIH (SEQ ID NO:4);
(2) HVR-H2 comprising the amino acid sequence AWISPYGGSTYYADSVKG (SEQ ID NO:5);
(3) HVR-H3 comprising the amino acid sequence WPGGFDY (SEQ ID NO:6). In some embodiments, the monoclonal antibody in the formulation is in a concentration of about 120 g/L to about 130 g/L. In some embodiments, the monoclonal antibody in the formulation is in a concentration of about 125 g/L. In some embodiments, the histidine acetate is in a concentration of about 17 mM to about 22 mM. In some embodiments, the histidine acetate is in a concentration of about 20 mM. In some embodiments, the sucrose is in a concentration of about 220 mM to about 260 mM. In some embodiments, the sucrose is in a concentration of about 240 mM. In some embodiments, the pH is about 5.5. In some embodiments, the polysorbate in the formulation is polysorbate 20. In some embodiments, the polysorbate is in a concentration of about 0.02% (w/v). In some embodiments, the formulation is mixed with a hyaluronidase enzyme prior to being administered to a subject. In some embodiments, the hyaluronidase enzyme is recombinant human hyaluronidase (rHuPH20). In some embodiments, the hyaluronidase enzyme concentration in the mixture is about 1000 U/ml to about 3000 U/ml. In some embodiments, the hyaluronidase enzyme concentration in the mixture is about 2000 U/ml.

In some embodiments of either of the aspects described above or any of the embodiments described herein, the monoclonal antibody is not subject to prior lyophilization. In some embodiments of either of the aspects described above or any of the embodiments described herein, the monoclonal antibody is a humanized antibody. In some embodiments of either of the aspects described above or any of the embodiments described herein, the monoclonal antibody comprises a light chain variable region comprising the amino acid sequence of SEQ ID NO:7, and a heavy chain variable region comprising the amino acid sequence of SEQ ID NO:8. In some embodiments of either of the aspects described above or any of the embodiments described herein, the monoclonal antibody is a full length antibody. In some embodiments of either of the aspects described above or any of the embodiments described herein, the monoclonal antibody is an IgG1 antibody. In some embodiments of either of the aspects described above or any of the embodiments described herein, the monoclonal antibody comprises a light chain comprising the amino acid sequence of SEQ ID NO:9, and a heavy comprising the amino acid sequence of SEQ ID NO: 10. In some embodiments of either of the aspects described above or any of the embodiments described herein, the monoclonal antibody is stored in a glass vial or a metal alloy container. In some embodiments of either of the aspects described above or any of the embodiments described herein, the metal alloy is 316L stainless steel or hastelloy. In some embodiments of either of the aspects described above or any of the embodiments described herein, the formulation is stable at 2-8° C. for at least 6 months. In some embodiments of either of the aspects described above or any of the embodiments described herein, the formulation is stable at 2-8° C. for at least 12 months. In some embodiments of either of the aspects described above or any of the embodiments described herein, the formulation is stable at 2-8° C. for at least 24 months. In some embodiments of either of the aspects described above or any of the embodiments described herein, the antibody in the formulation retains at least about 80% of its biological activity after storage. In some embodiments of either of the aspects described above or any of the embodiments described herein, the biological activity is measured by antibody binding to PD-L1. In some embodiments of either of the aspects described above or any of the embodiments described herein, the formulation is sterile. In some embodiments of either of the aspects described above, or any of the embodiments described herein the formulation is suitable to be administered to a subject. In some embodiments of either of the aspects described above or any of the embodiments described herein, the formulation is for subcutaneous administration.

Also provided herein is an article of manufacture comprising a container holding the liquid pharmaceutical formulation of any of the aspects or embodiments described above. In some embodiments, the container is a glass vial or a metal alloy container. In some embodiments, the metal alloy is 316L stainless steel or hastelloy.

Also provided herein is a kit comprising a container holding the liquid pharmaceutical formulation of any of the aspects or embodiments described above.

Also provided herein is a method of treating a disease or disorder in a subject comprising administering an effective amount of the liquid pharmaceutical formulation of any of the aspects or embodiments described above to the subject, wherein the disease or disorder is selected from the group consisting of infection, cancer, and inflammatory disease. In some embodiments, the disease or disorder is cancer. In some embodiments, the cancer is selected from the group consisting of non-small cell lung cancer, small cell lung cancer, urothelial carcinoma, and breast cancer. In some embodiments, the breast cancer is triple negative breast cancer. In some embodiments, the subject is a human.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-1C shows the levels of high molecular weight species (HMWS) (FIG. 1A), ion exchange chromatography (IEC) main peak percentages (FIG. 1B), and non-reducing capillary electrophoresis-SDS (NR CE-SDS) pre-peak sums (FIG. 1C) of various drug substance (DS) formulations after multiple freeze/thaw cycles.

FIG. 2A-2C shows the levels of acidic species (FIG. 2A), basic species (FIG. 2B) and HMWS (FIG. 2C) of various DS formulations after up to 1 month at 25° C.

FIG. 3A-3B shows the levels of HMWS (FIG. 3A) and SEC main peak percentages (FIG. 3B) of drug product (DP) formulations after up to 3 months at 25° C.

FIG. 4A-4B shows the levels of acidic species (FIG. 4A) and basic species (FIG. 4B) in DP formulations after up to 3 months at 25° C.

FIG. 5A-5B shows the percentages of pre-peaks (FIG. 5A) and NR CE-SDS main peak (FIG. 5B) in DP formulations after up to 3 months at 25° C.

FIG. 6A-6C shows the levels of HMWS (FIG. 6A), SEC main peak percentages (FIG. 6B), and NR CE-SDS sum of pre-peaks (FIG. 6C) in DP formulations after up to 1 month at 40° C.

FIG. 7A-7C shows the levels of the levels of acidic species (FIG. 7A), basic species (FIG. 7B), and percentage of IEC main peak (FIG. 7C) in DP formulations after up to 1 month at 40° C.

FIG. 8A-8B shows the stability of polysorbate 20 at 40° C. (FIG. 8A) and at 25° C. (FIG. 8B) for up to 3 months in various DP formulations.

FIG. 9A-9B shows rHuPH20 activity assays with various DP formulations at 25° C. for up to 3 months.

FIG. 10A-10B shows rHuPH20 activity in formulations comprising different concentrations of polysorbate in the while being agitate for 24 hours. Higher concentrations of polysorbate maintained rHuPH20 activity at higher levels under agitation at room temperature.

FIG. 11 shows the viscosities of various DP formulations at temperatures between 5° C. and 25° C.

### DETAILED DESCRIPTION

### I. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of aspects and embodiments.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

A “sterile” formulation is asceptic or free or essentially free from all living microorganisms and their spores.

A “frozen” formulation is one at a temperature below 0° C. Generally, the frozen formulation is not freeze-dried, nor is it subjected to prior, or subsequent, lyophilization. In certain embodiments, the frozen formulation comprises frozen drag substance for storage (in stainless steel tank) or frozen drug product (in final vial configuration).

A “stable” formulation is one in which the protein therein essentially retains its physical stability and/or chemical stability and/or biological activity upon storage. In some embodiments, the formulation essentially retains its physical and chemical stability, as well as its biological activity upon storage. The storage period is generally selected based on the intended shelf-life of the formulation. Various analytical techniques for measuring protein stability are available in the arc and are reviewed in Peptide and Protein Drug Delivery, 247-301, Vincent Lee Ed., Marcel Dekker, Inc., New York, N.Y., Pubs. (1991) and Jones, A. Adv. Drug Deliver}′ Rev. 10: 29-90 (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectromeiric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomeriation), clipping/hydrolysis/fragmentation (e.g., hinge region fragmentation), succinimide formation, impaired cysteine(s), N-terminal extension, C-terminal processing, giycosylation differences, etc.

A protein “retains its physical stability” in a pharmaceutical formulation if it shows no signs or very little of aggregation, precipitation and/or denaturation upon visual examination of color and/or clarity, or as measured by UV light scattering or by size exclusion chromatography.

A protein “retains its chemical stability” in a pharmaceutical formulation, if the chemical stability at a given time is such that the protein is considered to still retain its biological activity as defined below. Chemical stability can be assessed by detecting and quantifying chemically altered forms of the protein. Chemical alteration may involve size modification (e.g. clipping) which can be evaluated using size exclusion chromatography, SDS-PAGE and/or matrix-assisted laser desorption ionization/time-of-flight mass spectrometry (MALDI TOF MS), for example. Other types of chemical alteration include charge alteration (e.g. occurring as a result of deamidation) which can be evaluated by ion-exchange chromatography or icIEF, for example.

An antibody “retains its biological activity” in a pharmaceutical formulation, if the biological activity of the antibody at a given time is at least about 60% (within the errors of the assay) of the biological activity exhibited at the time the pharmaceutical formulation was prepared as determined in an assay (e.g., an antigen binding assay). Other “biological activity” assays for antibodies are elaborated herein below.

As used herein, “biological activity” of a monoclonal antibody includes the ability of the antibody to bind to antigen and resulting in a measurable biological response which can be measured in vitro or in vivo.

A “deamidated” monoclonal antibody herein is one in which one or more asparagine residue thereof has been derivitized, e.g. to an aspartic acid or an iso-aspartic acid.

An “oxidized” monoclonal antibody herein is one in which one or more tryptophan residue and/or one or more methionine thereof has been oxidized.

A “glycated” monoclonal antibody herein is one in which one or more lysine residue thereof has been glycated.

An antibody which is “susceptible to deamidation” is one comprising one or more residue, which has been found to be prone to deamidate.

An antibody which is “susceptible to oxidation” is one comprising one or more residue, which has been found to be prone to oxidize.

An antibody which is “susceptible to aggregation” is one which has been found to aggregate with other antibody molecule(s), especially upon freezing and/or agitation.

An antibody which is “susceptible to fragmentation” is one which has been found to be cleaved into two or more fragments, for example at a hinge region thereof.

By “reducing deamidation, oxidation, aggregation, or fragmentation” is intended preventing or decreasing the amount of deamidation, oxidation, aggregation, or fragmentation relative to the monoclonal antibody formulated in a different formulation.

The antibody which is formulated may be essentially pure and desirably essentially homogeneous (e.g., free from contaminating proteins etc.). “Essentially pure” antibody means a composition comprising at least about 90% by weight of the antibody, based on total weight of proteins in the composition, preferably at least about 95% by weight, “Essentially homogeneous” antibody means a composition comprising at least about 99% by weight of antibody, based on total weight of proteins in the composition.

By “isotonic” is meant that the formulation of interest has essentially the same osmotic pressure as human blood. Isotonic formulations generally have an osmotic pressure from about 250 to 350 mOsm. Isotonicity can be measured using a vapor pressure or ice-freezing type osmometer, for example.

As used herein, “buffer” refers to a buffered solution that resists changes in pH by the action of its acid-base conjugate components. In some embodiments, the buffer of this invention has a pH in the range from about 4.5 to about 7.0, preferably from about 5.6 to about 7.0, for example from 5.6 to 6.9, 5.7 to 6.8, 5.8 to 6.7, 5.9 to 6.6, 5.9 to 6.5, 6.0, 6.0 to 6.4, or 6.1 to 6.3. In one embodiment the buffer has a pH 5.6, 5.7, 5.8, 5.9, 6.0, 6.1, 6.2, 6.3, 6.4, 6.5, 6.6, 6.7, 6.8, 6.9, or 7.0. For example, sodium phosphate is an example of buffers chat will control the pH in this range.

As used herein, a “surfactant” refers to a surface-active agent, such as a nonionic surfactant. Examples of surfactants herein include polysorbate (for example, polysorbate 20 and, polysorbate 80); poloxamer (e.g. poloxamer 188); Triton; sodium dodecyl sulfate (SDS); sodium laurel sulfate; sodium octyi glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamido propyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.); polyethyl glycol, polypropyl glycol, and copolymers of ethylene and propylene glycol (e.g. Piuronics, PF68 etc); etc. In one embodiment, the surfactant herein is polysorbate 20.

In a pharmacological sense, in the context of the invention, a “therapeutically effective amount” of an antibody refers to an amount effective in the prevention or treatment of a disorder for the treatment of which the antibody is effective. A “disorder” is any condition that would benefit from treatment with the antibody. This includes chronic and acute disorders or diseases including those pathological conditions which predispose the mammal to the disorder in question.

A “preservative” is a compound which can be optionally included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. In one embodiment, the preservative herein is benzyl alcohol.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “effective amount” is at least the minimum amount required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells: reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drag, compound, or pharmaceutical composition. Thus, an “‘effective amount” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during, or after administration of the other treatment modality to the individual.

A “disorder” is any condition that would benefit from treatment including, but not limited to, chronic and acute disorders or diseases including those pathological conditions which predispose the mammal to the disorder in question.

The terms “cell proliferative disorder” and “proliferative disorder” refer to disorders that are associated with some degree of abnormal cell proliferation. In one embodiment, the cell proliferative disorder is cancer. In one embodiment, the cell proliferative disorder is a tumor.

“Tumor,” as used herein, refers to all neoplastic ceil growth and proliferation, whether malignant or benign, and all pre-cancerous and cancerous cells and tissues, The terms “cancer”, “cancerous”, “ceil proliferative disorder”, “proliferative disorder” and “tumor” are not mutually exclusive as referred to herein.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia or lymphoid malignancies. More particular examples of such cancers include, but not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL: high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as chat associated with brain tumors), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, mesothelioma, and multiple myeloma. In some embodiments, the cancer is selected from: small cell lung cancer, glioblastoma, neuroblastoma, melanoma, breast carcinoma, gastric cancer, colorectal cancer (CRC), and hepatocellular carcinoma. In some embodiments, the cancer is selected from: non-small cell lung cancer, colorectal cancer, glioblastoma and breast carcinoma, including metastatic forms of those cancers.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®, Bristol-Myers Squibb Oncology, Princeton, N.J.), albumin-engineered nanoparticle formulation of paclitaxel (ABRAXANE™), and docetaxel (TAXOTERE®, Rhome-Poulene Rorer, Antony, France); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors; tyrosine kinase inhibitors; serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin, and pharmaceutically acceptable salts, acids or derivatives of any of the above; as well as combinations of two or more of the above.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON.cndot.toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; Vinorelbine and Esperamicins (see U.S. Pat. No. 4,675,187), and pharmaceutically acceptable salts, acids or derivatives of any of the above; as well as combinations of two or more of the above.

A “growth inhibitory agent” when used herein refers to a compound or composition which inhibits growth of a cell either in vitro or in vivo. In one embodiment, growth inhibitory agent is growth inhibitory antibody that prevents or reduces proliferation of a cell expressing an antigen to which the antibody binds. In another embodiment, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

A “subject” or an “individual” for purposes of treatment refers to any animal classified as a mammal, including humans, domestic and farm animals, and zoo, sports, or pet animals, such as dogs, horses, cats, cows, etc. In some embodiments, the mammal is human.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the C_{H}1, C_{H}2 and C_{H}3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“K”) and lambda (“X.”), based on the amino acid sequences of their constant domains.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen binding region thereof. In some embodiments, the antibody fragment described herein is an antigen-binding fragment. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthün, in The Pharmacology of Monoclonal Antibodies, vol. 113, Rosenburg and Moore eds., (Springer-Verlag, New York, 1994), pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “species-dependent antibody” is one which has a stronger binding affinity for an antigen from a first mammalian species than it has for a homologue of that antigen from a second mammalian species. Normally, the species-dependent antibody “binds specifically” to a human antigen (e.g., has a binding affinity (Kd) value of no more than about 1×10⁻⁷ M, no more than about 1×10⁻⁸ M, or no more than about 1×10⁻⁹ M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above, and may be a humanized or human antibody.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat Numbering) |
| H1 | H31-H35 | H26-H35 | H26-H32 | H30-H35 |
| | | | | (Chothia Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG₁ EU antibody.

The expression “linear antibodies” refers to the antibodies described in Zapata et al. (1995 Protein Eng, 8(10):1057-1062). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

As use herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

### II. Antibody Formulations and Preparations

In some embodiments, provided herein are liquid pharmaceutical formulations comprising anti-PD-L1 antibodies as described herein, such as liquid pharmaceutical formulations for subcutaneous administration. In some embodiments, the formulation comprises an anti-PD-L1 antibody (e.g., monoclonal antibody), sucrose, a buffer, and a surfactant, wherein the formulation has a pH of about 5.0 to about 6.5. In some embodiments, the formulation further comprises methionine. In some embodiments, the anti-PD-L1 antibody described herein in the formulation is in a concentration of about 100 g/L to about 150 g/L. In some embodiments, the buffer is histidine (e.g., histidine acetate). In some embodiments, the buffer in the formulation is in a concentration of about 15 mM to about 25 mM. In some embodiments, sucrose in the formulation is about 200 mM to about 280 mM. In some embodiments, the surfactant in the formulation is polysorbate (e.g, polysorbate 20). In some embodiments, polysorbate in the formulation is in a concentration of about 0.005% (w/v) to about 0.08% (w/v). In some embodiments, the formulation comprises methionine in a concentration of about 5 mM to about 15 mM. In some embodiments, the formulation has a pH of about 5.0 to about 6.3. In some embodiments, provided herein is a liquid pharmaceutical formulations, the formulation comprising an anti-PD-L1 antibody as described herein in a concentration of about 100 g/L to about 150 g/L, histidine acetate in a concentration of about 15 mM to about 25 mM, sucrose in a concentration of about 200 mM to about 280 mM, polysorbate in a concentration of about 0.04% (w/v) to about 0.08% (w/v), methionine in a concentration of about 5 mM to about 15 mM, and pH of about 5.6 to about 6.0. In some embodiments, the formulation further comprises a hyaluronidase enzyme (e.g., recombinant human hyaluronidase (rHuPh20)). In some embodiments, the formulation comprises a hyaluronidase enzyme (e.g., rHuPh20) in a concentration of about 1000 U/ml to about 3000 U/ml. In some embodiments, the formulation is sterile. In some embodiments, the formulation is suitable to be administered to a subject. In some embodiments, the formulation is for subcutaneous administration.

In some embodiments, provided herein is a liquid pharmaceutical formulations, the formulation comprising an anti-PD-L1 antibody as described herein in a concentration of about 100 g/L to about 150 g/L, histidine acetate in a concentration of about 15 mM to about 25 mM, sucrose in a concentration of about 200 mM to about 280 mM, polysorbate in a concentration of about 0.01% (w/v) to about 0.03% (w/v), and pH of about 5.3 to about 5.7. In some embodiments, the formulation is sterile. In some embodiments, the formulation is suitable to be administered to a subject. In some embodiments, the formulation is for subcutaneous administration.

In some embodiments, the antibody in the formulation is stable at −20° C. for at least about 6 months, at least about 12 months, at least about 18 months, at least two years, at least three years, or at least four years. In some embodiments, the antibody in the formulation is stable at 2-8° C. for at least about 6 months, at least about 12 months, at least about 18 months, at least two years, or at least three years. In some embodiments, after storage, the antibody retains at least about 60%, at least about 65%, at least about 70%, at least about 75%, at least about 80%, at least about 85%, at least about 90%, or at least about 95% of its biological activity (e.g., binding to the target, or therapeutic potency) exhibited before storage, i.e., at the time the pharmaceutical formulation was prepared.

In certain embodiments, the formulation is stable at about 40° C. for at least about 1, 2, 3, 4, 5, 6, 7, 14, 21, 28, or more days. In certain embodiments, the formulation is stable at about 40° C. for at least about 1, 2, 3, 4, 5, 6, 7, 8, or more weeks. In certain embodiments, the formulation is stable at about 25° C. for at least 1, 2, 3, 4, 5, 6, 7, 8, 9, 10, 11, 12, 13, 14, 15, 16, 17, 18, 19, 20, 21, 22, 23, 24, or more months. In certain embodiments, the formulation is stable at about 5° C. for at least 1, 2, 3, 4, 5, 6, 7, 8, 9, 10, 11, 12, 13, 14, 15, 16, 17, 18, 19, 20, 21, 22, 23, 24, or more months. In certain embodiments, the formulation is stable at about −20° C. for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more months. In certain embodiments, the formulation is stable at 5° C. or −20° C. for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more months. Furthermore, in some embodiments the formulation is stable following freezing (to, e.g., −20° C., −40° C. or −70° C.) and thawing of the formulation, for example following 1, 2 3, 4, or 5 cycles of freezing and thawing.

A. Anti-PD-L1 Antibodies

In some embodiments, the antibody in the formulation is an anti-PD-L1 antibody. PD-L1 (programmed cell death 1 ligand 1), also known as PDL1, B7-H1, B7-4, CD274, and B7-H, is a transmembrane protein, and its interaction with PD-1 inhibits T-cell activation and cytokine production. In some embodiments, the anti-PD-L1 antibody described herein binds to human PD-L1. Examples of anti-PDL1 antibodies that can be formulated using the formulations described herein are described in PCT patent application WO 2010/077634 A1, U.S. Pat. No. 8,217,149, and US 2016/0319022 which are incorporated herein by reference.

In some embodiments, the anti-PD-L1 antibody is capable of inhibiting binding between PD-L1 and PD-1 and/or between PD-L1 and B7-1. In some embodiments, the anti-PD-L1 antibody is a monoclonal antibody. In some embodiments, the anti-PD-L1 antibody is an antibody fragment selected from the group consisting of Fab, Fab′-SH, Fv, scFv, and (Fab′)₂ fragments. In some embodiments, the anti-PD-L1 antibody is a full-length antibody. In some embodiments, the anti-PD-L1 antibody is a humanized antibody. In some embodiments, the anti-PD-L1 antibody is a human antibody.

Anti-PD-L1 antibodies described in WO 2010/077634 A1, U.S. Pat. No. 8,217,149, and US 2016/0319022 may be formulated in the formulations described herein.

In some embodiments, the anti-PD-L1 antibody in a formulation described herein comprises:

(a) a light chain variable region comprising:
(1) HVR-L1 comprising the amino acid sequence RASQDVSTAVA (SEQ ID NO:1);
(2) HVR-L2 comprising the amino acid sequence SASFLYS (SEQ ID NO:2);
(3) HVR-L3 comprising the amino acid sequence QQYLYHPAT (SEQ ID NO:3); and

(b) a heavy chain variable region comprising:
(1) HVR-H1 comprising the amino acid sequence GFTFSDSWIH (SEQ ID NO:4);
(2) HVR-H2 comprising the amino acid sequence AWISPYGGSTYYADSVKG (SEQ ID NO:5);
(3) HVR-H3 comprising the amino acid sequence WPGGFDY (SEQ ID NO:6).

In a still further embodiment, the anti-PD-L1 antibody in a formulation described herein comprises a heavy chain and a light chain sequence, wherein:

(a) the heavy chain variable region sequence has at least 85% sequence identity to the heavy chain variable region sequence:

(SEQ ID NO: 8)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS,

or

(b) the light chain variable region sequence has at least 85% sequence identity to the light chain variable region sequence:

(SEQ ID NO: 7)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIK.

In some embodiments, said monoclonal antibody in the formulation comprises a light chain variable region comprising the amino acid sequence of SEQ ID NO:7, and a heavy chain variable region comprising the amino acid sequence of SEQ ID NO:8. In some embodiments, said monoclonal antibody in the formulation comprises a light chain variable region having at least 85%, 86%, 87%, 88%, 89%, 90%, 91%, 92%, 93%, 94%, 95%, 96%, 97%, 98%, or 99% sequence identity to the light chain variable region having the amino acid sequence of SEQ ID NO:7, and a heavy chain variable region having at least 85%, 86%, 87%, 88%, 89%, 90%, 91%, 92%, 93%, 94%, 95%, 96%, 97%, 98%, or 99% identity to the heavy chain variable region having the amino acid sequence of SEQ ID NO:8.

In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region if IgG2A. In a still further specific aspect, the antibody has reduced or minimal effector function. In a still further specific aspect the minimal effector function results from an “effector-less Fc mutation” or aglycosylation. In still a further embodiment, the effector-less Fc mutation is an N297A or D265A/N297A substitution in the constant region.

In a still further embodiment, the anti-PD-L1 antibody in a formulation described herein comprises a heavy chain and a light chain sequence, wherein:

(a) the heavy chain sequence has at least 85% sequence identity to the heavy chain sequence:

(SEQ ID NO: 10)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG,

or

(b) the light chain sequences has at least 85% sequence identity to the light chain sequence:

(SEQ ID NO: 9)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In some embodiments, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO:9. In some embodiments, provided is an isolated anti-PD-L1 antibody comprising a heavy chain and a light chain sequence, wherein the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO:10. In some embodiments, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO:9 and the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO:10.

In some embodiments, provided is an isolated anti-PD-L1 antibody comprising a heavy chain and a light chain, wherein the light chain comprises the amino acid sequence of SEQ ID NO:9 and the heavy chain comprises the amino acid sequence of SEQ ID NO:10.

In some embodiments, the anti-PD-L1 antibody in a formulation described herein comprises:

(a) a light chain variable region comprising:
(1) HVR-L1 comprising the amino acid sequence RASQDVSTAVA (SEQ ID NO:1);
(2) HVR-L2 comprising the amino acid sequence SASFLYS (SEQ ID NO:2);
(3) HVR-L3 comprising the amino acid sequence QQYLYHPAT (SEQ ID NO:3); and

(b) a heavy chain variable region comprising:
(1) HVR-H1 comprising the amino acid sequence GFTFSDSWIH (SEQ ID NO:4);
(2) HVR-H2 comprising the amino acid sequence AWISPYGGSTYYADSVKG (SEQ ID NO:5);
(3) HVR-H3 comprising the amino acid sequence WPGGFDY (SEQ ID NO:6). In some embodiments, the anti-PD-L1 antibody comprises a light chain variable region comprising the amino acid sequence of SEQ ID NO:7, and a heavy chain variable region comprising the amino acid sequence of SEQ ID NO:8. In some embodiments, said monoclonal antibody in the formulation comprises a light chain variable region having at least 85%, 86%, 87%, 88%, 89%, 90%, 91%, 92%, 93%, 94%, 95%, 96%, 97%, 98%, or 99% sequence identity to the light chain variable region having the amino acid sequence of SEQ ID NO:7, and a heavy chain variable region having at least 85%, 86%, 87%, 88%, 89%, 90%, 91%, 92%, 93%, 94%, 95%, 96%, 97%, 98%, or 99% identity to the heavy chain variable region having the amino acid sequence of SEQ ID NO:8.

In some embodiments, the anti-PD-L1 antibody in a formulation described herein comprises:

(a) a light chain variable region comprising:
(1) HVR-L1 comprising the amino acid sequence RASQDVSTAVA (SEQ ID NO:1);
(2) HVR-L2 comprising the amino acid sequence SASFLYS (SEQ ID NO:2);
(3) HVR-L3 comprising the amino acid sequence QQYLYHPAT (SEQ ID NO:3); and

(b) a heavy chain variable region comprising:
(1) HVR-H1 comprising the amino acid sequence GFTFSDSWIH (SEQ ID NO:4);
(2) HVR-H2 comprising the amino acid sequence AWISPYGGSTYYADSVKG (SEQ ID NO:5);
(3) HVR-H3 comprising the amino acid sequence WPGGFDY (SEQ ID NO:6). In some embodiments, the anti-PD-L1 antibody comprises a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO:9. In some embodiments, provided is an isolated anti-PD-L1 antibody comprising a heavy chain and a light chain sequence, wherein the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO:10. In some embodiments, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO:9 and the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO:10.

In some embodiments, the isolated anti-PD-L1 antibody is an oxidized monoclonal antibody. In some embodiments, the oxidized monoclonal antibody in the formulation comprises a light chain comprising the amino acid sequence of SEQ ID NO:9, and a heavy comprising the amino acid sequence of SEQ ID NO:10. In some embodiments, the oxidized monoclonal antibody in the formulation comprises a heavy chain comprising the amino acid sequence of SEQ ID NO:10, wherein one or more of W33, W50, or W101 is oxidized. In some embodiments, the oxidized monoclonal antibody in the formulation comprises a heavy chain comprising the amino acid sequence of SEQ ID NO:10, wherein one or more of M253 and M429 is oxidized. In some embodiments, the oxidized monoclonal antibody retains at least about 60%, at least about 65%, at least about 70%, at least about 75%, at least about 80%, at least about 85%, at least about 90%, or at least about 95% of its biological activity (e.g., binding to the target, or therapeutic potency) exhibited before storage, i.e., at the time the pharmaceutical formulation was prepared.

In some embodiments, the isolated anti-PD-L1 antibody is a glycated monoclonal antibody. In some embodiments, the glycated monoclonal antibody in the formulation comprises a light chain comprising the amino acid sequence of SEQ ID NO:9, and a heavy comprising the amino acid sequence of SEQ ID NO:10. In some embodiments, the glycated monoclonal antibody in the formulation comprises a heavy chain comprising the amino acid sequence of SEQ ID NO:10, wherein one or more of lysine is glycated. In some embodiments, the glycated monoclonal antibody in the formulation comprises a heavy chain comprising the amino acid sequence of SEQ ID NO:10, wherein K65 is glycated.

In some embodiments, the isolated anti-PD-L1 antibody is aglycosylated.

In some embodiments, the anti-PD-L1 antibody is atezolizumab (TECENTRIQ®).

In any of the embodiments herein, the isolated anti-PDL1 antibody can bind to a human PD-L1, for example a human PD-L1 as shown in UniProtKB/Swiss-Prot Accession No. Q9NZQ7.1, or a variant thereof.

In a still further embodiment, provided is an isolated nucleic acid encoding any of the antibodies described herein. In some embodiments, the nucleic acid further comprises a vector suitable for expression of the nucleic acid encoding any of the previously described anti-PD-L1 antibodies. In a still further specific aspect, the vector is in a host cell suitable for expression of the nucleic acid. In a still further specific aspect, the host cell is a eukaryotic cell or a prokaryotic cell. In a still further specific aspect, the eukaryotic cell is a mammalian cell, such as Chinese Hamster Ovary (CHO).

The antibody or antigen binding fragment thereof, may be made using methods known in the art, for example, by a process comprising culturing a host cell containing nucleic acid encoding any of the previously described anti-PD-L1 antibodies or antigen-binding fragment in a form suitable for expression, under conditions suitable to produce such antibody or fragment, and recovering the antibody or fragment.

B. Antibody Preparation

In general, various methodologies for preparing antibodies for use in research, testing, and clinical are well-established in the art. The antibody in the formulation is prepared using techniques available in the art for generating antibodies, exemplary methods of which are described in WO 2010/077634 A1, U.S. Pat. No. 8,217,149, and US 2016/0319022.

C. Biologically Active Antibodies

Antibodies produced as described above may be subjected to one or more “biological activity” assays to select an antibody with beneficial properties from a therapeutic perspective or selecting formulations and conditions that retain biological activity of the antibody. The antibody may be tested for its ability to bind the antigen against which it was raised. For example, for an anti-PD-L1 antibody, the antigen binding properties of the antibody can be evaluated in an assay that detects the ability to bind to PD-L1. In some embodiments, the binding of the antibody may be determined by saturation binding; ELISA; and/or competition assays (e.g. RIA's), for example. Also, the antibody may be subjected to other biological activity assays, e.g., in order to evaluate its effectiveness as a therapeutic. Such assays are known in the art and depend on the target antigen and intended use for the antibody. For example, the biological effects of PD-L1 blockade by the antibody can be assessed in CD8+ T cells, a lymphocytic choriomeningitis virus (LCMV) mouse model and/or a syngeneic tumor model e.g., as described in U.S. Pat. No. 8,217,149.

To screen for antibodies which bind to a particular epitope on the antigen of interest (e.g., those which block binding of the anti-PDL1 antibody of the example to PD-L1), a routine cross-blocking assay such as that described in Antibodies, A Laboratory Manual, Cold Spring Harbor Laboratory, Ed Harlow and David Lane (1988), can be performed. Alternatively, epitope mapping, e.g. as described in Champe et al., J. Biol. Chem. 270:1388-1394 (1995), can be performed to determine whether the antibody binds an epitope of interest.

D. Preparation of the Formulation

After preparation of the antibody of interest (e.g., techniques for producing antibodies which can be formulated as disclosed herein will be elaborated below and are known in the art), the pharmaceutical formulation comprising it is prepared. In certain embodiments, the antibody to be formulated has not been subjected to prior lyophilization and the formulation of interest herein is an aqueous formulation. In certain embodiments, the formulation is for subcutaneous administration. In certain embodiments, the antibody is a full length antibody. In one embodiment, the antibody in the formulation is an antibody fragment, such as an F(ab′)₂, in which case problems that may not occur for the full length antibody (such as clipping of the antibody to Fab) may need to be addressed. The therapeutically effective amount of antibody present in the formulation is determined by taking into account the desired dose volumes and mode(s) of administration, for example. From about 100 g/L to about 150 g/L, or from about 110 g/L to about 140 g/L, or from about 120 g/L to about 130 g/L is an exemplary concentration of an antibody as described herein in the formulation. In some embodiments, the antibody in the formulation is in a concentration of about 100 g/L to about 150 g/L. In some embodiments, the antibody in the formulation is in a concentration of about 110 g/L to about 140 g/L. In some embodiments, the antibody in the formulation is in a concentration of about 120 g/L to about 130 g/L. In some embodiments, the antibody in the formulation is in a concentration of about 100 g/L. In some embodiments, the antibody in the formulation is in a concentration of about 105 g/L. In some embodiments, the antibody in the formulation is in a concentration of about 110 g/L. In some embodiments, the antibody in the formulation is in a concentration of about 115 g/L. In some embodiments, the antibody in the formulation is in a concentration of about 120 g/L. In some embodiments, the antibody in the formulation is in a concentration of about 125 g/L. In some embodiments, the antibody in the formulation is in a concentration of about 130 g/L. In some embodiments, the antibody in the formulation is in a concentration of about 135 g/L. In some embodiments, the antibody in the formulation is in a concentration of about 140 g/L. In some embodiments, the antibody in the formulation is in a concentration of about 145 g/L. In some embodiments, the antibody in the formulation is in a concentration of about 150 g/L. From 100 g/L to 150 g/L, or from 110 g/L to 140 g/L, or from 120 g/L to 130 g/L is an exemplary concentration of an antibody as described herein in the formulation. In some embodiments, the antibody in the formulation is in a concentration of 100 g/L to 150 g/L. In some embodiments, the antibody in the formulation is in a concentration of 110 g/L to 140 g/L. In some embodiments, the antibody in the formulation is in a concentration of 120 g/L to 130 g/L. In some embodiments, the antibody in the formulation is in a concentration of 100 g/L. In some embodiments, the antibody in the formulation is in a concentration of 105 g/L. In some embodiments, the antibody in the formulation is in a concentration of 110 g/L. In some embodiments, the antibody in the formulation is in a concentration of 115 g/L. In some embodiments, the antibody in the formulation is in a concentration of 120 g/L. In some embodiments, the antibody in the formulation is in a concentration of 125 g/L. In some embodiments, the antibody in the formulation is in a concentration of 130 g/L. In some embodiments, the antibody in the formulation is in a concentration of 135 g/L. In some embodiments, the antibody in the formulation is in a concentration of 140 g/L. In some embodiments, the antibody in the formulation is in a concentration of 145 g/L. In some embodiments, the antibody in the formulation is in a concentration of 150 g/L.

A liquid pharmaceutical formulation is prepared comprising the antibody in a pH-buffered solution. The buffer of this invention has a pH in the range from about 5.0 to about 6.5. In certain embodiments the pH is in the range from about 5.3 to about 6.0, the pH is in the range from about 5.6 to about 6.0, in the range from about 5.7 to about 5.9, the pH is in the range from about 5.3 to about 5.7, the pH is in the range from about 5.4 to about 5.6, the pH is in the range from about 5.5 to about 5.8, the pH is in the range from about 5.0 to about 6.0, the pH is in the range from about 5.1 to about 5.8, the pH is in the range from about 5.2 to about 5.8, the pH is in the range from about 5.3 to about 5.8, or the pH is in the range from about 5.4 to about 5.8. In certain embodiments of the invention, the formulation has a pH of 5.2 or about 5.2. In certain embodiments of the invention, the formulation has a pH of 5.3 or about 5.3. In certain embodiments of the invention, the formulation has a pH of 5.4 or about 5.4. In certain embodiments of the invention, the formulation has a pH of 5.5 or about 5.5. In certain embodiments of the invention, the formulation has a pH of 5.6 or about 5.6. In certain embodiments of the invention, the formulation has a pH of 5.7 or about 5.7. In certain embodiments of the invention, the formulation has a pH of 5.8 or about 5.8. In certain embodiments of the invention, the formulation has a pH of 5.9 or about 5.9. In certain embodiments of the invention, the formulation has a pH of 6.0 or about 6.0. Examples of buffers that will control the pH within this range include histidine (such as L-histidine) or sodium acetate. In certain embodiments, the buffer contains histidine acetate or sodium acetate in the concentration of about 15 mM to about 25 mM. In certain embodiments of the invention, the buffer contains histidine acetate or sodium acetate in the concentration of about 15 mM to about 25 mM, about 16 mM to about 25 mM, about 17 mM to about 25 mM, about 18 mM to about 25 mM, about 19 mM to about 25 mM, about 20 mM to about 25 mM, about 21 mM to about 25 mM, about 22 mM to about 25 mM, about 15 mM, about 16 mM, about 17 mM, about 18 mM, about 19 mM, about 20 mM, about 21 mM, about 22 mM, about 23 mM, about 24 mM, or about 25 mM. In certain embodiments, the buffer contains histidine acetate in the concentration of about 15 mM to about 25 mM. In certain embodiments of the invention, the buffer contains histidine acetate in the concentration of about 15 mM to about 25 mM, about 16 mM to about 25 mM, about 17 mM to about 25 mM, about 18 mM to about 25 mM, about 19 mM to about 25 mM, about 20 mM to about 25 mM, about 21 mM to about 25 mM, about 22 mM to about 25 mM, about 15 mM, about 16 mM, about 17 mM, about 18 mM, about 19 mM, about 20 mM, about 21 mM, about 22 mM, about 23 mM, about 24 mM, or about 25 mM. In certain embodiments of the invention, the buffer contains histidine acetate in the concentration of about 20 mM. In one embodiment, the buffer is histidine acetate in an amount of about 20 mM, pH 5.0. In one embodiment, the buffer is histidine acetate in an amount of about 20 mM, pH 5.1. In one embodiment, the buffer is histidine acetate in an amount of about 20 mM, pH 5.2. In one embodiment, the buffer is histidine acetate in an amount of about 20 mM, pH 5.3. In one embodiment, the buffer is histidine acetate in an amount of about 20 mM, pH 5.4. In one embodiment, the buffer is histidine acetate in an amount of about 20 mM, pH 5.5. In one embodiment, the buffer is histidine acetate in an amount of about 20 mM, pH 5.6. In one embodiment, the buffer is histidine acetate in an amount of about 20 mM, pH 5.7. In one embodiment, the buffer is histidine acetate in an amount of about 20 mM, pH 5.8. In one embodiment, the buffer is histidine acetate in an amount of about 20 mM, pH 5.9. In one embodiment, the buffer is histidine acetate in an amount of about 20 mM, pH 6.0. In one embodiment, the buffer is histidine acetate in an amount of about 20 mM, pH 6.1. In one embodiment, the buffer is histidine acetate in an amount of about 20 mM, pH 6.2. In one embodiment, the buffer is histidine acetate in an amount of about 20 mM, pH 6.3. In one embodiment, the buffer is histidine acetate in an amount of about 20 mM, pH 6.4. In one embodiment, the buffer is histidine acetate in an amount of about 20 mM, pH 6.5. In one embodiment, the buffer is histidine acetate in an amount of 20 mM, pH 5.0. In one embodiment, the buffer is histidine acetate in an amount of 20 mM, pH 5.1. In one embodiment, the buffer is histidine acetate in an amount of 20 mM, pH 5.2. In one embodiment, the buffer is histidine acetate in an amount of 20 mM, pH 5.3. In one embodiment, the buffer is histidine acetate in an amount of 20 mM, pH 5.4. In one embodiment, the buffer is histidine acetate in an amount of 20 mM, pH 5.5. In one embodiment, the buffer is histidine acetate in an amount of 20 mM, pH 5.6. In one embodiment, the buffer is histidine acetate in an amount of 20 mM, pH 5.7. In one embodiment, the buffer is histidine acetate in an amount of 20 mM, pH 5.8. In one embodiment, the buffer is histidine acetate in an amount of 20 mM, pH 5.9. In one embodiment, the buffer is histidine acetate in an amount of 20 mM, pH 6.0. In one embodiment, the buffer is histidine acetate in an amount of 20 mM, pH 6.1. In one embodiment, the buffer is histidine acetate in an amount of 20 mM, pH 6.2. In one embodiment, the buffer is histidine acetate in an amount of 20 mM, pH 6.3. In one embodiment, the buffer is histidine acetate in an amount of 20 mM, pH 6.4. In one embodiment, the buffer is histidine acetate in an amount of 20 mM, pH 6.5.

The formulation further comprises sucrose in an amount of about 200 mM to about 280 mM. In some embodiments, sucrose in the formulation is about 210 mM to about 280 mM, about 220 mM to about 280 mM, about 230 mM to about 280 mM, about 240 mM to about 280 mM, about 200 mM to about 270 mM, about 200 mM to about 260 mM, about 200 mM to about 240 mM, about 210 mM to about 270 mM, about 220 mM to about 260 mM, about 230 mM to about 250 mM, or about 235 mM to about 245 mM. In some embodiments, sucrose in the formulation is about 200 mM, about 210 mM, about 220 mM, about 230 mM, about 235 mM, about 240 mM, about 245 mM, about 250 mM, about 260 mM, about 270 mM, or about 280 mM. In some embodiments, sucrose in the formulation is about 240 mM. The formulation further comprises sucrose in an amount of 200 mM to 280 mM. In some embodiments, sucrose in the formulation is 210 mM to 280 mM, 220 mM to 280 mM, 230 mM to 280 mM, 240 mM to 280 mM, 200 mM to 270 mM, 200 mM to 260 mM, 200 mM to 240 mM, 210 mM to 270 mM, 220 mM to 260 mM, 230 mM to 250 mM, or 235 mM to 245 mM. In some embodiments, sucrose in the formulation is 200 mM, 210 mM, 220 mM, 230 mM, about 235 mM, 240 mM, 245 mM, 250 mM, 260 mM, 270 mM, or 280 mM. In some embodiments, sucrose in the formulation is 240 mM.

In some embodiments, a surfactant is added to the antibody formulation. Exemplary surfactants include nonionic surfactants such as polysorbates (e.g. polysorbates 20, 80 etc) or poloxamers (e.g. poloxamer 188, etc.). The amount of surfactant added is such that it reduces aggregation of the formulated antibody and/or minimizes the formation of particulates in the formulation and/or reduces adsorption. For example, the surfactant may be present in the formulation in an amount from about 0.005% (w/v) to about 0.08% (w/v). In some embodiments, the surfactant (e.g., polysorbate 20) is from about 0.005% to about 0.07%, from about 0.005% to about 0.065%, from about 0.005% to about 0.06%, from about 0.01% to about 0.08%, from about 0.015% to about 0.08%, from about 0.02% to about 0.08%, from about 0.01% to about 0.03%, from about 0.01% to about 0.025%, from about 0.01% to about 0.02%, from about 0.015% to about 0.03%, from about 0.02% to about 0.03%, from about 0.015% to about 0.025%, from about 0.02% to about 0.04%, from about 0.05% to about 0.08%, from about 0.055% to about 0.08%, from about 0.06% to about 0.08%, from about 0.05% to about 0.07%, from about 0.05% to about 0.065%, from about 0.055% to about 0.065%, from about 0.06% to about 0.07%, from about 0.06% to about 0.065%, from about 0.055% to about 0.06%, or from about 0.055% to about 0.07%. In certain embodiments, the surfactant (e.g., polysorbate 20) is about 0.02% (w/v). In certain embodiments, the surfactant (e.g., polysorbate 20) is about 0.06% (w/v). In certain embodiments, the surfactant (e.g., polysorbate 20) is 0.02% (w/v). In certain embodiments, the surfactant (e.g., polysorbate 20) is 0.06% (w/v). In certain embodiments, the surfactant (e.g., polysorbate 20) is present in the formulation in an amount of 0.01% or about 0.01%. In certain embodiments, the surfactant (e.g., polysorbate 20) is present in the formulation in an amount of 0.015% or about 0.015%. In certain embodiments, the surfactant (e.g., polysorbate 20) is present in the formulation in an amount of 0.02% or about 0.02%. In certain embodiments, the surfactant (e.g., polysorbate 20) is present in the formulation in an amount of 0.025% or about 0.025%. In certain embodiments, the surfactant (e.g., polysorbate 20) is present in the formulation in an amount of 0.03% or about 0.03%. In certain embodiments, the surfactant (e.g., polysorbate 20) is present in the formulation in an amount of 0.05% or about 0.05%. In certain embodiments, the surfactant (e.g., polysorbate 20) is present in the formulation in an amount of 0.055% or about 0.055%. In certain embodiments, the surfactant (e.g., polysorbate 20) is present in the formulation in an amount of 0.06% or about 0.06%. In certain embodiments, the surfactant (e.g., polysorbate 20) is present in the formulation in an amount of 0.065% or about 0.065%. In certain embodiments, the surfactant (e.g., polysorbate 20) is present in the formulation in an amount of 0.07% or about 0.07%.

In some embodiments, methionine is added to the antibody formulation. In some embodiments, methionine in the formulation is about 1 mM to about 20 mM, about 5 mM to about 15 mM, about 6 mM to about 14 mM, about 7 mM to about 13 mM, about 8 mM to about 12 mM, about 9 mM to about 11 mM, about 8 mM to about 13 mM, about 8 mM to about 11 mM, about 8 mM to about 10 mM, about 9 mM to about 13 mM, about 9 mM to about 12 mM, or about 9 mM to about 10 mM. In certain embodiments, methionine in the formulation is about 5 mM, about 6 mM, about 7 mM, about 8 mM, about 9 mM, about 10 mM, about 11 mM, about 12 mM, about 13 mM, about 14 mM or about 15 mM. In a particular embodiment, methionine in the formulation is about 10 mM. In some embodiments, methionine in the formulation is 1 mM to 20 mM, 5 mM to 15 mM, 6 mM to 14 mM, 7 mM to 13 mM, 8 mM to 12 mM, 9 mM to 11 mM, 8 mM to 13 mM, 8 mM to 11 mM, 8 mM to 10 mM, 9 mM to 13 mM, 9 mM to 12 mM, or 9 mM to 10 mM. In certain embodiments, methionine in the formulation is 5 mM, 6 mM, 7 mM, 8 mM, 9 mM, 10 mM, 11 mM, 12 mM, 13 mM, 14 mM or 15 mM. In a particular embodiment, methionine in the formulation is 10 mM.

In certain embodiments, a hyaluronan-degrading enzyme (or hyaluronidase) or hyaluronan synthesis inhibitor is added to an antibody formulation described herein, is mixed with an antibody formulation described herein prior to administration, or is co-administered with an antibody formulation described herein. Hyaluronan (hyaluronic acid; HA) is a glycosaminoglycan that exists predominantly in connective tissues, skin, cartilage, and in synovial fluid in mammals. In connective tissue, the water of hydration associated with hyaluronan creates hydrated matrices between tissues. HA is found in the extracellular matrix of many cells, especially in soft connective tissues. Hyaluronidases are enzymes that degrade hyaluronan.

Glycosaminoglycans (GAGs) are complex linear polysaccharides of the extracellular matrix (ECM). GAGS are characterized by repeating disaccharide structures of an N-substituted hexosamine and an uronic acid (in the case of hyaluronan (HA), chondroitin sulfate (CS), chondroitin (C), dermatan sulfate (DS), heparan sulfate (HS), and heparin (H)), or a galactose (in the case of keratan sulfate (KS)). Except for HA, all exist covalently bound to core proteins. The GAGS with their core proteins are structurally referred to as proteoglycans (PGs).

HA is found in the extracellular matrix of many cells, especially in soft connective tissues. HA has been assigned various physiological functions, such as in water and plasma protein homeostasis (Laurent T. C. et al, FASEB J., 1992; 6: 2397-2404). HA production increases in proliferating cells and may play a role in mitosis. It has also been implicated in locomotion and cell migration. HA seems to play important roles in cell regulation, development, and differentiation (Laurent et al, supra). HA has widely been used in clinical medicine. Its tissue protective and rheological properties have proved useful in ophthalmic surgery (e.g. to protect the corneal endothelium during cataract surgery). Hyaluronan protein interactions also are involved in the structure of the extracellular matrix or “ground substance”.

Hyaluronidases are a group of generally neutral- or acid-active enzymes found throughout the animal kingdom. Hyaluronidases vary with respect to substrate specificity, and mechanism of action (WO 2004/078140). There are three general classes of hyaluronidases:

1. Mammalian-type hyaluronidases, (EC 3.2.1.35) which are endo-beta-N-acetylhexosamimdases with tetrasaccharides and hexasaccharides as the major end products. They have both hydrolytic and transglycosidase activities, and can degrade hyaluronan and chondroitin sulfates (CS), generally C4-S and C6-S.

2. Bacterial hyaluronidases (EC 4.2.99.1) degrade hyaluronan and, and to various extents, CS and DS. They are endo-beta-N-acetylhexosaminidases that operate by a beta elimination reaction that yields primarily disaccharide end products.

3. Hyaluronidases (EC 3.2.1.36) from leeches, other parasites, and crustaceans are endo-beta-glucuronidases that generate tetrasaccharide and hexasaccharide end products through hydrolysis of the beta 1-3 linkage.

Mammalian hyaluronidases can be further divided into two groups: neutral-active and acid-active enzymes. There are six hyaluronidase-like genes in the human genome, HYAL1, HYAL2, HYAL3, HYAL4, HYALP1 and PH20/SPAM1. HYALP1 is a pseudogene, and HYAL3 has not been shown to possess enzyme activity toward any known substrates. HYAL4 is a chondroitinase and exhibits little activity towards hyaluronan. HYAL1 is the prototypical acid-active enzyme and PH20 is the prototypical neutral-active enzyme. Acid-active hyaluronidases, such as HYAL1 and HYAL2 generally lack catalytic activity at neutral pH (i.e. pH 7). For example, HYAL1 has little catalytic activity in vitro over pH 4.5 (Frost I. G. and Stern, R., “A microtiter-based assay for hyaluronidase activity not requiring specialized reagents”, Anal. Biochemistry, 1997; 251:263-269). HYAL2 is an acid-active enzyme with a very low specific activity in vitro.

The hyaluronidase-like enzymes can also be characterized by those which are generally locked to the plasma membrane via a glycosylphosphatidyl inositol anchor such as human HYAL2 and human PH20 (Danilkovitch-Miagkova et al, Proc. Natl. Acad. Sci. USA, 2003; 100(8):4580-4585; Phelps et al, Science 1988; 240(4860): 1780-1782), and those which are generally soluble such as human HYAL1 (Frost, I. G. et al, “Purification, cloning, and expression of human plasma hyaluronidase”, Biochem. Biophys. Res. Commun. 1997; 236(1): 10-15). However, there are variations from species to species: bovine PH20 for example is very loosely attached to the plasma membrane and is not anchored via a phospho lipase sensitive anchor (Lalancette et al, Biol Reprod., 2001; 65(2):628-36). This unique feature of bovine hyaluronidase has permitted the use of the soluble bovine testes hyaluronidase enzyme as an extract for clinical use (Wydase™, Hyalase™). Other PH20 species are lipid anchored enzymes that are generally not soluble without the use of detergents or lipases. For example, human PH20 is anchored to the plasma membrane via a GPI anchor. Attempts to make human PH20 DNA constructs that would not introduce a lipid anchor into the polypeptide resulted in either a catalytically inactive enzyme, or an insoluble enzyme (Arming et al, Eur. J. Biochem., 1997; 1; 247(3):810-4). Naturally occurring macaque sperm hyaluronidase is found in both a soluble and membrane bound form. While the 64 kDa membrane bound form possesses enzyme activity at pH 7.0, the 54 kDa form is only active at pH 4.0 (Cherr et al, Dev. Biol, 1996; 10; 175(1): 142-53). Thus, soluble forms of PH20 are often lacking enzyme activity under neutral conditions.

In accordance with the teachings in WO2006/091871 and U.S. Pat. No. 7,767,429, small amounts of soluble hyaluronidase glycoproteins (sHASEGPs) can be introduced into a formulation in order to facilitate the administration of therapeutic drug into the hypodermis. By rapidly depolymerizing HA in the extracellular space sHASEGP reduces the viscosity of the interstitium, thereby increasing hydraulic conductance and allowing for larger volumes to be administered safely and comfortably into the SC tissue. The increased hydraulic conductance induced by sHASEGP through reduced interstitial viscosity allows for greater dispersion, potentially increasing the systemic bioavailability of SC administered therapeutic drug.

When injected in the hypodermis, the depolymerization of HA by sHASEGP is localized to the injection site in the SC tissue. Experimental evidence shows that the sHASEGP is inactivated locally in the interstitial space with a half-life of 13 to 20 minutes in mice, without detectable systemic absorption in blood following single intravenous dose in CD-I mice. Within the vascular compartment sHASEGP demonstrates a half-life of 2.3 and 5 minutes in mice and Cynomolgus monkeys, respectively, with doses up to 0.5 mg/kg. The rapid clearance of sHASEGP, combined with the continual synthesis of the HA substrate in the SC tissue, results in a transient and locally-active permeation enhancement for other co-injected molecules, the effects of which are fully reversible within 24 to 48 hours post administration (Bywaters G. L., et al, “Reconstitution of the dermal barrier to dye spread after Hyaluronidase injection”, Br. Med. J., 1951; 2 (4741): 1178-1183).

In addition to its effects on local fluid dispersion, sHASEGP also acts as absorption enhancer. Macromolecules greater than 16 kilodaltons (kDa) are largely excluded from absorption through the capillaries via diffusion and are mostly absorbed via the draining lymph nodes. A subcutaneously administered macromolecule such as e.g. a therapeutic antibody (molecular weight approximately 150 kDa) must therefore traverse the interstitial matrix before reaching the draining lymphatics for subsequent absorption into the vascular compartment. By increasing local dispersion, sHASEGP increases the rate (Ka) of absorption of many macromolecules. This leads to increased peak blood levels (Cₘₐₓ) and potentially to increased bioavailability relative to SC administration in the absence of sHASEGP (Bookbinder L. H., et al, “A recombinant human enzyme for enhanced interstitial transport of therapeutics”, J. Control. Release 2006; 114: 230-241).

Hyaluronidase products of animal origin have been used clinically for over 60 years, primarily to increase the dispersion and absorption of other co-administered drugs and for hypodermoclysis (SC injection/infusion of fluid in large volume) (Frost G. I., “Recombinant human hyaluronidase (rHuPH20): an enabling platform for subcutaneous drug and fluid administration”, Expert Opinion on Drug Delivery, 2007; 4: 427-440). The details on the mechanism of action of hyaluronidases have been described in detail in the following publications: Duran-Reynolds F., “A spreading factor in certain snake venoms and its relation to their mode of action”, CR Soc Biol Paris, 1938; 69-81; Chain E., “A mucolytic enzyme in testes extracts”, Nature 1939; 977-978; Weissmann B., “The transglycosylative action of testicular hyaluronidase”, J. Biol. Chem., 1955; 216: 783-94; Tammi, R., Saamanen, A. M., Maibach, H. I., Tamrni M., “Degradation of newly synthesized high molecular mass hyaluronan in the epidermal and dermal compartments of human skin in organ culture”, J. Invest. Dermatol. 1991; 97: 126-130; Laurent, U. B. G., Dahl, L. B., Reed, R. K., “Catabolism of hyaluronan in rabbit skin takes place locally, in lymph nodes and liver”, Exp. Physiol. 1991; 76: 695-703; Laurent, T. C. and Fraser, J. R. E., “Degradation of Bioactive Substances: Physiology and Pathophysiology”, Henriksen, J. H. (Ed) CRC Press, Boca Raton, Fla.; 1991. pp. 249-265; Harris, E. N., et al, “Endocytic function, glycosaminoglycan specificity, and antibody sensitivity of the recombinant human 190-kDa hyaluronan receptor for endocytosis (HARE)”, J. Biol. Chem. 2004; 279:36201-36209; Frost, G. I., “Recombinant human hyaluronidase (rHuPH20): an enabling platform for subcutaneous drug and fluid administration”, Expert Opinion on Drug Delivery, 2007; 4: 427-440. Hyaluronidase products approved in EU countries include Hylase® “Dessau” and Hyalase®. Hyaluronidase products of animal origin approved in the US include Vitrase™ Hydase™, and Amphadase™.

The safety and efficacy of hyaluronidase products have been widely established. The most significant safety risk identified is hypersensitivity and/or allergenicity, which is thought to be related to the lack of purity of the animal-derived preparations (Frost, G. I., “Recombinant human hyaluronidase (rHuPH20): an enabling platform for subcutaneous drug and fluid administration”, Expert Opinion on Drug Delivery, 2007; 4: 427-440). It should be noted that there are differences with respect to the approved dosages of animal-derived hyaluronidases between the UK, Germany and the US. In the UK, the usual dose as an adjuvant to subcutaneous or intramuscular injection is 1500 units, added directly to the injection. In the US, the usual dose used for this purpose is 150 units. In hypodermoclysis, hyaluronidase is used to aid the subcutaneous administration of relatively large volumes of fluids. In the UK, 1500 units of hyaluronidase are generally given with each 500 to 1000 ml of fluid for subcutaneous use. In the US, 150 units are considered adequate for each liter of hypodermoclysis solution. In Germany, 150 to 300 units are considered adequate for this purpose. In the UK, the diffusion of local anesthetics is accelerated by the addition of 1500 units. In Germany and the US 150 units are considered adequate for this purpose. The dosage differences notwithstanding (the dosage in the UK is ten times higher than in the US), no apparent differences in the safety profiles of animal-derived hyaluronidase products marketed in the US and UK, respectively, have been reported. On Dec. 2, 2005, Halozyme Therapeutics Inc. received approval from the FDA for an injectable formulation of the recombinant human hyaluronidase, rHuPH20 (HYLENEX™). The FDA approved HYLENEX™ at a dose of 150 units for SC administration of the following indications:
as an adjuvant to increase the absorption and dispersion of other injected drugs
for hypodermoclysis
as an adjunct in SC urography for improving resorption of radiopaque agents.

As part of that regulatory review it was established that rHuPH20 possesses the same properties of enhancing the dispersion and absorption of other injected drugs as the previously approved animal-derived hyaluronidase preparations, but with an improved safety profile. In particular, the use of recombinant human hyaluronidase (rHuPH20) compared with animal-derived hyaluronidases minimizes the potential risk of contamination with animal pathogens and transmissible spongiform encephalopathies.

Soluble Hyaloronidase glycoproteins (sHASEGP), a process for preparing the same and their use in pharmaceutical compositions have been described in WO 2004/078140. The detailed experimental work as outlined further below has shown that the claimed formulation surprisingly has favorable storage stability and fulfils all necessary requirements for approval by the health authorities.

The hyaluronidase enzyme in the formulation in accordance with the present invention is believed to enhance the delivery of the anti-PD-L1 antibody to the systemic circulation, e.g. by increasing the absorption of the active substance (it acts as a permeation enhancer). The hyaluronidase enzyme is also believed to increases the delivery of the therapeutic anti-anti-PD-L1 antibody into the systemic circulation via the subcutaneous application route by the reversible hydrolyzation of hyaluronan, an extracellular component of the SC interstitial tissue. The hydrolysis of hyaluronan in the hypodermis temporarily opens channels in the interstitial space of the SC tissue and thereby improves the delivery of the therapeutic anti-PD-L1 antibody into the systemic circulation. In addition, the administration shows reduced pain in humans and less volume-derived swelling of the SC tissue.

Hyaluronidase, when administered locally has its entire effect locally. In other word hyaluronidase is inactivated and metabolized locally in minutes and has not been noted to have systemic or long term effects. The rapid inactivation of hyaluronidase within minutes when it enters the blood stream precludes a realistic ability to perform comparable bio distribution studies between different hyaluronidase products. This property also minimizes any potential systemic safety concerns because the hyaluronidase product cannot act at distant sites. The unifying feature of all hyaluronidase enzymes in accordance with the present invention is their ability to depolymerize hyaluronan, regardless of differences in chemical structure, in species source, in tissue sources, or in the batches of drug product sourced from the same species and tissue. They are unusual in the fact that their activity is the same (except for potency) in spite of having different structures. The hyaluronidase enzyme in accordance with the formulation of the present invention is characterized by having no adverse effect on the molecular integrity of the anti-PD-L1 antibody in the stable pharmaceutical formulation described herein. Furthermore, the hyaluronidase enzyme merely modifies the delivery of the anti-PD-L1 antibody to the systemic circulation but does not possess any properties that could provide or contribute to the therapeutic effects of systemically absorbed anti-PD-L1 antibody. The hyaluronidase enzyme is not systemically bioavailable and does not adversely affect the molecular integrity of the anti-PD-L1 antibody at the recommended storage conditions of the stable pharmaceutical formulation in accordance with the invention. It is therefore to be considered as an excipient in the anti-PD-L1 antibody formulation in accordance with this invention. As it exerts no therapeutic effect it represents a constituent of the pharmaceutical form apart from the therapeutically active anti-PD-L1 antibody. A number of suitable hyaluronidase enzymes in accordance with the present invention are known from the prior art. In some embodiments, the enzyme is a human hyaluronidase enzyme, such as the enzyme known as rHuPH20. rHuPH20 is a member of the family of neutral and acid-active β-1,4 glycosyl hydrolases that depolymerize hyaluronan by the hydrolysis of the β-1,4 linkage between the Ci position of N-acetyl glucosamine and the C₄ position of glucuronic acid. Hyaluronan is a polysaccharide found in the intracellular ground substance of connective tissue, such as the subcutaneous interstitial tissue, and of certain specialized tissues, such as the umbilical cord and vitreous humor. The hydrolysis of hyaluronan temporarily decreases the viscosity of the interstitial tissue and promotes the dispersion of injected fluids or of localized transudates or exudates, thus facilitating their absorption. The effects of hyaluronidase are local and reversible with complete reconstitution of the tissue hyaluronan occurring within 24 to 48 hours (Frost, G. I., “Recombinant human hyaluronidase (rHuPH20): an enabling platform for subcutaneous drug and fluid administration”, Expert Opinion on Drug Delivery, 2007; 4:427-440). The increase in the permeability of connective tissue through the hydrolysis of hyaluronan correlates with the efficacy of hyaluronidase for their capability to increase the dispersion and absorption of co-administered molecules.

The human genome contains several hyaluronidase genes. Only the PH20 gene product possesses effective hyaluronidase activity under physiologic extracellular conditions and acts as a spreading agent, whereas acid-active hyaluronidases do not have this property. rHuPH20 is the first and only recombinant human hyaluronidase enzyme currently available for therapeutic use. The human genome contains several hyaluronidase genes; only the PH20 gene product possesses effective hyaluronidase activity under physiologic extracellular conditions and acts as a spreading agent. Naturally occurring human PH20 protein has a lipid anchor attached to the carboxy terminal amino acid that anchors it to the plasma membrane. The rHuPH20 enzyme developed by Halozyme is a truncated deletion variant that lacks such amino acids in the carboxy terminus responsible for the lipid attachment. This gives rise to a soluble, neutral pH-active enzyme similar to the protein found in bovine testes preparations. The rHuPH20 protein is synthesized with a 35 amino acid signal peptide that is removed from the N-terminus during the process of secretion. The mature rHuPH20 protein contains an authentic N-terminal amino acid sequence orthologous to that found in some bovine hyaluronidase preparations.

The PH20 hyaluronidases, including the animal derived PH20 and recombinant human rHuPH20, depolymerize hyaluronan by the hydrolysis of the β-1,4 linkage between the Ci position of N-acetyl glucosamine and the C4 position of glucuronic acid. The tetrasaccharide is the smallest digestion product (Weissmann, B., “The transglycosylative action of testicular hyaluronidase”, J. Biol. Chem., 1955; 216: 783-94). This N-acetyl glucosamine/glucuronic acid structure is not found in N-linked glycans of recombinant biological products and therefore rHuPH20 will not affect the glycosylation of antibodies it is formulated with. The rHuPH20 enzyme itself possesses six N-linked glycans per molecule with core structures similar to that found in monoclonal antibodies. As anticipated, these N-linked structures do not change over time, confirming the lack of enzymatic activity of rHuPH20 on these N-linked glycan structures. The short half-life of rHuPH20 and the constant synthesis of hyaluronan lead to a short and local action of the enzyme on tissues.

The hyaluronidase enzyme which is an excipient in the subcutaneous formulation in accordance with the present invention is may be prepared by using recombinant DNA technology. In this way it is ensured that the same protein (identical amino acid sequence) is obtained all the time and that allergic reactions caused by contaminating proteins co-purified during extraction from a tissue is avoided. In some embodiments, the hyaluronidase enzyme used in the formulation in accordance with the present invention is a human enzyme, such as rHuPH20. The amino acid sequence of rHuPH20 (HYLENEX™) is well known and available under CAS Registry No. 757971-58-7. The approximate molecular weight is 61 kDa (see also U.S. Pat. No. 7,767,429).

Multiple structural and functional comparisons have been performed between naturally sourced mammalian hyaluronidase and PH-20 cDNA clones from humans and other mammals. The PH-20 gene is the gene used for the recombinant product rHuPH20; however the recombinant drug product is a 447 amino acid truncated version of the full protein encoded by the PH-20 gene. Structural similarities with respect to amino acid sequences rarely exceed 60% in any comparison. Functional comparisons show that the activity of rHuPH20 is very similar to that of previously approved hyaluronidase products. This information is consistent with the clinical findings during the past 50 years that regardless of the source of the hyaluronidase, the clinical safety and efficacy of units of hyaluronidase are equivalent. The use of rHuPH20 in the anti-PD-L1 antibody SC formulation in accordance with the present invention allows the administration of higher volumes of drug product and to potentially enhance the absorption of subcutaneously administered anti-PD-L1 antibody, such as atezolizumab, into the systemic circulation.

It has been proposed to facilitate the subcutaneous injection of therapeutic proteins and antibodies by using small amounts of soluble hyaluronidase glycoproteins (sHASEGPs); see WO2006/091871. It has been shown that the addition of such soluble hyaluronidase glycoproteins (either as a combined formulation or by co-administration) facilitates the administration of therapeutic drug into the hypodermis. By rapidly depolymerizing hyaluronan HA in the extracellular space sHASEGP reduces the viscosity of the interstitium, thereby increasing hydraulic conductance and allowing for larger volumes to be administered safely and comfortably into the subcutaneous tissue. The increased hydraulic conductance induced by sHASEGP through reduced interstitial viscosity allows for greater dispersion, potentially increasing the systemic bioavailability of SC administered therapeutic drug.

In some embodiments, a formulation described herein comprises an effective amount of at least one hyaluronidase enzyme (e.g. rHuPH20), such as in an amount from about 1000 U/ml to about 5000 U/ml. In some embodiments, the hyaluronidase enzyme (e.g., rHuPH20) is present in the formulation at a concentration of about 1000 U/ml to about 4000 U/ml. In some embodiments, the hyaluronidase enzyme (e.g., rHuPH20) is present in the formulation at a concentration of about 1000 U/ml to about 3000 U/ml. In some embodiments, the hyaluronidase enzyme (e.g., rHuPH20) is present in the formulation at a concentration of about 1000 U/ml to about 2000 U/ml. In some embodiments, the hyaluronidase enzyme (e.g., rHuPH20) is present in the formulation at a concentration of about 2000 U/ml to about 4000 U/ml. In some embodiments, the hyaluronidase enzyme (e.g., rHuPH20) is present in the formulation at a concentration of about 2000 U/ml to about 3000 U/ml. In some embodiments, the hyaluronidase enzyme (e.g., rHuPH20) is present in the formulation at a concentration of about 1500 U/ml to about 3000 U/ml. In some embodiments, the hyaluronidase enzyme (e.g., rHuPH20) is present in the formulation at a concentration of about 1500 U/ml to about 2500 U/ml. In some embodiments, the hyaluronidase enzyme (e.g., rHuPH20) is present in the formulation at a concentration of about 1500 U/ml to about 2000 U/ml. In some embodiments, the hyaluronidase enzyme (e.g., rHuPH20) is present in the formulation at a concentration of about 2000 U/ml to about 2500 U/ml. In some embodiments, the hyaluronidase enzyme (e.g., rHuPH20) is present in the formulation at a concentration of about 1750 U/ml to about 2250 U/ml. In some embodiments, the hyaluronidase enzyme (e.g., rHuPH20) is present in the formulation at a concentration of about 1900 U/ml to about 2100 U/ml. In some embodiments, the hyaluronidase enzyme (e.g., rHuPH20) is present in the formulation at a concentration of about 1950 U/ml to about 2050 U/ml. In some embodiments, the hyaluronidase enzyme (e.g., rHuPH20) is present in the formulation at a concentration of about 2000 U/ml. In some embodiments, the hyaluronidase enzyme (e.g., rHuPH20) is present in the formulation at a concentration of 1000 U/ml to 4000 U/ml. In some embodiments, the hyaluronidase enzyme (e.g., rHuPH20) is present in the formulation at a concentration of 1000 U/ml to 3000 U/ml. In some embodiments, the hyaluronidase enzyme (e.g., rHuPH20) is present in the formulation at a concentration of 1000 U/ml to 2000 U/ml. In some embodiments, the hyaluronidase enzyme (e.g., rHuPH20) is present in the formulation at a concentration of 2000 U/ml to 4000 U/ml. In some embodiments, the hyaluronidase enzyme (e.g., rHuPH20) is present in the formulation at a concentration of 2000 U/ml to 3000 U/ml. In some embodiments, the hyaluronidase enzyme (e.g., rHuPH20) is present in the formulation at a concentration of 1500 U/ml to 3000 U/ml. In some embodiments, the hyaluronidase enzyme (e.g., rHuPH20) is present in the formulation at a concentration of 1500 U/ml to 2500 U/ml. In some embodiments, the hyaluronidase enzyme (e.g., rHuPH20) is present in the formulation at a concentration of 1500 U/ml to 2000 U/ml. In some embodiments, the hyaluronidase enzyme (e.g., rHuPH20) is present in the formulation at a concentration of 2000 U/ml to 2500 U/ml. In some embodiments, the hyaluronidase enzyme (e.g., rHuPH20) is present in the formulation at a concentration of 1750 U/ml to 2250 U/ml. In some embodiments, the hyaluronidase enzyme (e.g., rHuPH20) is present in the formulation at a concentration of 1900 U/ml to 2100 U/ml. In some embodiments, the hyaluronidase enzyme (e.g., rHuPH20) is present in the formulation at a concentration of 1950 U/ml to about 2050 U/ml. In some embodiments, the hyaluronidase enzyme (e.g., rHuPH20) is present in the formulation at a concentration of 2000 U/ml.

The liquid pharmaceutical formulations of the present invention comprising a hyaluronidase enzyme are particularly suited for subcutaneous injection. It is clearly understood by the person skilled in the art that such a formulation comprising an anti-PD-L1 antibody and a hyaluronidase enzyme can be provided for administration in form of one single combined formulation or alternatively in form of two separate formulations which can be mixed just prior to the subcutaneous injection. Alternatively the anti-PD-L1 antibody and the hyaluronidase enzyme can be administered as separate injections at different sites of the body, such as at sites which are immediately adjacent to each other. It is also possible to inject the therapeutic agents present in the formulation in accordance with the present invention as consecutive injections, e.g. first the hyaluronidase enzyme followed by the injection of the anti-anti-PD-L1 antibody formulation. These injections can also be performed in the reversed order, viz. by first injecting the anti-PD-L1 antibody formulation followed by injecting the hyaluronidase enzyme. In case the anti-PD-L1 antibody and the hyaluronidase enzyme are administered as separate injections, one or both of the proteins have to be provided with the buffering agent, the stabilizer(s) and the nonionic surfactant in the concentrations as specified in the appended claims but excluding the hyaluronidase enzyme. The hyaluronidase enzyme can then be provided e.g. in a L-histidine/HCl buffer at pH of about 6.5, 100 to 150 mM NaCl and 0.01 to 0.1% (w/v) polysorbate 20 or polysorbate 80. In one embodiment the anti-PD-L1 antibody is provided with the buffering agent, the stabilizer(s) and the nonionic surfactant in the concentrations as specified herein.

As noted above the hyaluronidase enzyme may be considered to be a further excipient in the anti-PD-L1 antibody formulation. The hyaluronidase enzyme may be added to the anti-PD-L1 antibody formulation at the time of manufacturing the anti-PD-L1 antibody formulation or may be added shortly before the injection. Alternatively the hyaluronidase enzyme may be provided as a separate injection. In the latter case the hyaluronidase enzyme may be provided in a separate vial either in lyophilized form which must be reconstituted with suitable diluents before the subcutaneous injection takes place, or may be provided as a liquid formulation by the manufacturer. The anti-PD-L1 antibody formulation and the hyaluronidase enzyme may be procured as separate entities or may also be provided as kits comprising both injection components and suitable instructions for their subcutaneous administration. Suitable instructions for the reconstitution and/or administration of one or both of the formulations may also be provided.

Therefore the present invention also provides pharmaceutical compositions consisting of an a highly concentrated, stable pharmaceutical formulation of a pharmaceutically active anti-PD-L1 antibody or a mixture of such antibody and a suitable amount of at least one hyaluronidase enzyme in the form of a kit comprising both injection components and suitable instructions for their subcutaneous administration.

A further aspect of the present invention relates to injection devices comprising a liquid pharmaceutical formulation in accordance with the present invention. Such formulation may consist of a pharmaceutically active anti-PD-L1 antibody or a mixture of such antibody molecules and suitable excipients as outlined herein and may additionally comprise a hyaluronidase enzyme either as a combined formulation or as a separate formulation for co-administration.

In some embodiments, provided herein is a liquid pharmaceutical formulation, the formulation comprising a monoclonal anti-PD-L1 antibody described herein in a concentration of about 100 g/L to about 150 g/L, histidine acetate in a concentration of about 15 mM to about 25 mM, sucrose in a concentration of about 200 mM to about 280 mM, polysorbate in a concentration of about 0.04% (w/v) to about 0.08% (w/v), methionine in a concentration of about 5 mM to about 15 mM, a hyaluronidase enzyme in a concentration of about 1000 U/ml to about 3000 U/ml, pH of about 5.6 to about 6.0. In some embodiments, the formulation is sterile. In some embodiments, the formulation is suitable to be administered to a subject. In some embodiments, the formulation is for subcutaneous administration.

In some embodiments, provided herein is a liquid pharmaceutical formulation, the formulation comprising a monoclonal anti-PD-L1 antibody described herein in a concentration of about 125 g/L, histidine acetate in a concentration of about 20 mM, sucrose in a concentration of about 240 mM, polysorbate 20 in a concentration of about 0.06% (w/v), methionine in a concentration of about 10 mM, rHuPH20 in a concentration of about 2000, and pH of about 5.8. In some embodiments, the formulation is sterile. In some embodiments, the formulation is suitable to be administered to a subject. In some embodiments, the formulation is for subcutaneous administration.

In some embodiments, provided herein is a liquid pharmaceutical formulation, the formulation comprising a monoclonal anti-PD-L1 antibody described herein in a concentration of about 100 g/L to about 150 g/L, histidine acetate in a concentration of about 15 mM to about 25 mM, sucrose in a concentration of about 200 mM to about 280 mM, polysorbate in a concentration of about 0.01% (w/v) to about 0.03% (w/v), and pH of about 5.3 to about 5.7. In some embodiments, the formulation is mixed with a hyaluronidase enzyme prior to being administered to a subject. In some embodiments, the hyaluronidase enzyme concentration in the mixture is about 1000 U/ml to about 3000 U/ml. In some embodiments, the formulation is sterile. In some embodiments, the formulation is suitable to be administered to a subject. In some embodiments, the formulation is for subcutaneous administration.

In some embodiments, provided herein is a liquid pharmaceutical formulation, the formulation comprising a monoclonal anti-PD-L1 antibody described herein in a concentration of about 125 g/L, histidine acetate in a concentration of about 20 mM, sucrose in a concentration of about 240 mM, polysorbate 20 in a concentration of about 0.02% (w/v), and pH of about 5.5. In some embodiments, the formulation is mixed with rHuPH20 prior to being administered to a subject. In some embodiments, the rHuPH20 concentration in the mixture is about 2000 U/. In some embodiments, the formulation is sterile. In some embodiments, the formulation is suitable to be administered to a subject. In some embodiments, the formulation is for subcutaneous administration.

In one embodiment, the formulation contains the above-identified agents (e.g., antibody, buffer, sucrose, and/or surfactant) and is essentially free of one or more preservatives, such as benzyl alcohol, phenol, m-cresol, chlorobutanol and benzethonium Cl. In another embodiment, a preservative may be included in the formulation, particularly where the formulation is a multidose formulation. The concentration of preservative may be in the range from about 0.1% to about 2%, such as from about 0.5% to about 1%. One or more other pharmaceutically acceptable carriers, excipients or stabilizers such as those described in Remington's Pharmaceutical Sciences 16th edition, Osol, A. Ed. (1980) may be included in the formulation provided that they do not adversely affect the desired characteristics of the formulation. Acceptable carriers, excipients or stabilizers are nontoxic to recipients at the dosages and concentrations employed and include; additional buffering agents; co-solvents; anti-oxidants including ascorbic acid and methionine; chelating agents such as EDTA; metal complexes (e.g. Zn-protein complexes); biodegradable polymers such as polyesters; and/or salt-forming counterions. Exemplary pharmaceutically acceptable carriers herein further include insterstitial drug dispersion agents such as soluble neutral-active hyaluronidase glycoproteins (sHASEGP), for example, human soluble PH20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The formulation herein may also contain more than one protein as necessary for the particular indication being treated, such as those with complementary activities that do not adversely affect the other protein. For example, where the antibody is anti-PD-L1, it may be combined with another agent (e.g., a chemotherapeutic agent, and anti-neoplastic agent).

In some embodiments, the physical stability, chemical stability, or biological activity of the antibody in the formulation is evaluated or measured. Any methods known in the art and described in the Examples herein may be used to evaluate the stability and biological activity of the antibody in the formulation. For example, stability of the antibody in the formulation can be measured by, but not limited to, size exclusion chromatography (SEC or SE-HPLC), imaged capillary isoelectric focusing (ICIEF), peptide mapping, small-volume light obscuration (HIAC) assay, and capillary electrophoresis (CE) techniques such as CE-sodium dodecyl sulfate (CE-SDS) and CE-glycan analysis. In some embodiments, the antibody in the formulation is stable at −20° C. for at least about 6 months, at least about 8 months, at least about 10 months, at least about 12 months, at least about 14 months, at least about 16 months, at least about 18 months, at least about 20 months, at least about 21 months, at least about 22 months, at least about 23 months, at least about 24 months, at least about 3 years, or at least about 4 years. In some embodiments, the antibody in the formulation is stable at 2° C. to 8° C. (e.g., 5° C.) for at least about 6 months, at least about 8 months, at least about 10 months, at least about 12 months, at least about 14 months, at least about 16 months, at least about 18 months, at least about 20 months, at least about 21 months, at least about 22 months, at least about 23 months, or at least about 24 months. In some embodiments, the stability of the antibody (i.e., an antibody monomer) is measured by size exclusion chromatography in the formulation after storage. In some embodiments, the stability of the antibody is (i.e., an antibody monomer) measured by imaged capillary isoelectric focusing in the formulation after storage. In some embodiments, the percent of antibody monomer in the formulation as compared to total protein (e.g., including antibody and aggregates) is greater than about 60%, about 65%, about 70%, about 75%, about 80%, about 85%, about 86%, about 87%, about 88%, about 89%, about 90%, about 91%, about 92%, about 93%, about 94% or about 95% after storage at −20° C. for at least about 6 months, at least about 12 months, at least about 18 months, or at least about 24 months. In some embodiments, the percent of antibody monomer in the formulation as compared to (e.g., including antibody and aggregates) is greater than about 60%, about 65%, about 70%, about 75%, about 80%, about 85%, about 86%, about 87%, about 88%, about 89%, about 90%, about 91%, about 92%, about 93%, about 94% or about 95% after storage at 2° C. to 8° C. (e.g., 5° C.) for at least about 6 months, at least about 12 months, at least about 18 months, or at least about 24 months. In some embodiments, the percent of antibody monomer in the formulation as compared to (e.g., including antibody and aggregates) is greater than about 60%, about 65%, about 70%, about 75%, about 80%, about 85%, about 86%, about 87%, about 88%, about 89%, about 90%, about 91%, about 92%, about 93%, about 94% or about 95% after agitation at room temperature (e.g., about 15° C. to 25° C.) for at least about 2 hours, at least about 4 hours, at least about 6 hours, at least about 8 hours, at least about 10 hours, at least about 12 hours, at least about 14 hours, at least about 16 hours, at least about 18 hours, at least about 20 hours, or at least about 24 hours. In some embodiments, the percent of total aggregates (e.g., high molecular weight species and low molecular weight species) in the formulation is less than any of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% after storage at −20° C. for at least about 6 months, at least about 12 months, at least about 18 months, or at least about 24 months. In some embodiments, the percent of total aggregates (e.g., high molecular weight species and low molecular weight species) in the formulation is less than any of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% after storage at 2° C. to 8° C. (e.g., 5° C.) for at least about 6 months, at least about 12 months, at least about 18 months, or at least about 24 months. In some embodiments, the percent of total aggregates (e.g., high molecular weight species and low molecular weight species) in the formulation is less than any of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% after agitation at room temperature (e.g., about 15° C. to 25° C.) for at least about 2 hours, at least about 4 hours, at least about 6 hours, at least about 8 hours, at least about 10 hours, at least about 12 hours, at least about 14 hours, at least about 16 hours, at least about 18 hours, at least about 20 hours, or at least about 24 hours. In any of the embodiments herein, the stable formulation can be stored in a glass vial, a metal alloy container, or an intravenous (IV) bag. In some embodiments, the metal alloy is 316L stainless steel or hastelloy.

The formulations to be used for in vivo administration should be sterile. This is readily accomplished by filtration through sterile filtration membranes, prior to, or following, preparation of the formulation.

### III. Methods of Treatment and Administration of Antibody Formulations

The formulation is administered to a mammal in need of treatment with the antibody, such as a human, in accord with known methods, such as intravenous administration (e.g., as a bolus or by continuous infusion over a period of time), by intramuscular, intraperitoneal, intracerobrospinal, subcutaneous, intra-articular, intrasynovial, intrathecal, oral, topical, or inhalation routes. In one embodiment, the formulation is administered to the mammal by intravenous administration. For such purposes, the formulation may be injected using a syringe or via an IV line, for example. In one embodiment, the formulation is administered to the mammal by subcutaneous administration.

The appropriate dosage (“therapeutically effective amount”) of the antibody will depend, for example, on the condition to be treated, the severity and course of the condition, whether the antibody is administered for preventive or therapeutic purposes, previous therapy, the patient's clinical history and response to the antibody, the type of antibody used, and the discretion of the attending physician. The antibody is suitably administered to the patient at one time or over a series of treatments and may be administered to the patient at any time from diagnosis onwards. The antibody may be administered as the sole treatment or in conjunction with other drugs or therapies useful in treating the condition in question.

As a general proposition, the therapeutically effective amount of the antibody administered to human will be in the range of about 0.01 to about 50 mg/kg of patient body weight whether by one or more administrations. In some embodiments, the antibody used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some embodiments, the antibody is administered at 15 mg/kg. However, other dosage regimens may be useful. In one embodiment, an anti-PD-L1 antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The dose of the antibody administered in a combination treatment may be reduced as compared to a single treatment. The progress of this therapy is easily monitored by conventional techniques.

The formulations containing anti-PD-L1 antibody described herein can be used in a variety of in vitro and in vivo diagnostic and therapeutic applications. For example, the formulation containing the antibody may be administered to a subject or an individual for treating a disease or disorder (e.g., disease or disorder mediated by the PD-1 and PD-L1 interaction).

In some embodiments, the disease or disorder is cancer. In some embodiments, the cancer is locally advanced or metastatic. In some embodiments, the cancer is selected from the group consisting of a solid tumor, a hematologic cancer, bladder cancer, brain cancer, breast cancer, colon cancer, colorectal cancer, gastric cancer, glioma, head cancer, leukemia, liver cancer, lung cancer (e.g., non-small cell lung cancer), lymphoma, myeloma, neck cancer, ovarian cancer, melanoma, pancreatic cancer, renal cancer, salivary cancer, stomach cancer, thymic epithelial cancer, thyroid cancer, and squamous cell carcinoma of the head and neck. In some embodiments, the cancer is non-small cell lung cancer. In some embodiments, the cancer is small cell lung cancer. In some embodiments, the cancer is urothelial carcinoma. In some embodiments, the cancer is breast cancer. In some embodiments, the breast cancer is triple negative breast cancer. In some embodiments, the subject or individual treated has PD-L1 positive cancer cells (e.g., detected by IHC).

In some embodiments, the disease or disorder is infection. In some embodiments, the infection is a persistent infection. In some embodiments, the infection is a viral infection, a bacterial infection, a fungal infection, a helminth infection, or a protozoan infection. In some embodiments, the viral infection is selected from the group consisting of cytomegalovirus Epstein-Barr virus, hepatitis B, hepatitis C virus, herpes virus, measles virus, influenza, human immunodeficiency virus, human T lymphotropic virus, lymphocytic choriomeningitis virus, respiratory syncytial virus, and/or rhinovirus. In some embodiments, the bacterial infection is selected from the group consisting of Helicobacter spp., Mycobacterium spp., Porphyromonas spp., Chlamydia spp., Salmonella spp., Listeria spp., Streptococcus spp., Haemophilus spp., Neisseria spp., Klebsiella spp., Borrelia spp., Bacterioides spp., and Treponema spp. In some embodiments, the protozoan infection is selected from the group consisting of Leishmania spp., Plasmodium falciparum, Schistosoma spp., Toxoplasma spp., Trypanosoma spp., and Taenia spp. In some embodiments, the fungal infection is selected from the group consisting of blastomycosis, coccidioiodmycosis, histoplamsosis, candidiasis, cryptococcosis, aspergillossi, mucomycosis and pneumocystosis.

In some embodiments, the disease or disorder is an inflammatory disease. In some embodiments, the inflammatory disease is selected from the group consisting of acute disseminated encephalomyelitis, Addison's disease, Alzheimer's disease, ankylosing spondylitis, antiphospholipid antibody syndrome, atherosclerosis, autoimmune hemolytic anemia, autoimmune hepatitis, arthritis, Behcet's disease, Berger's disease, Bullous pemphigoid, Celiac disease, Chagas' disease, cholangitis, Crohn's disease, Dermatomyositis, Diabetes mellitus type 1, glomerulonephritis, Goodpasture's syndrome, graft-versus-host disease, Graves' disease, Guillain-Barré syndrome, Hashimoto's disease, hives, hyper IgE syndrome, idiopathic thrombocytopenic purpura, lupus erythematosus, lupus nephritis, multiple sclerosis, myasthenia gravis, organ transplant rejection, Parkinson's disease, pemphigus, pernicious anaemia, polymyositis, primary biliary cirrhosis, psoriasis, Raynaud's syndrome, rheumatoid arthritis, scleroderma, Sjögren's syndrome, temporal arteritis, thyroiditis, ulcerative colitis, uveitis, vasculitis, and Wegener's granulomatosis.

In some embodiments, the formulation containing the antibody may be administered in conjunction with another therapeutic agent to a subject or an individual for treating a disease or disorder. For example, for treating cancer, the anti-PD-L1 antibody formulation described herein may administered in conjunction with another anti-cancer treatment (e.g., a chemotherapy or a different antibody treatment).

### IV. Articles of Manufacture or Kits

In another embodiment of the invention, an article of manufacture or a kit is provided comprising a container which holds the liquid pharmaceutical formulation of the invention and optionally provides instructions for its use. Suitable containers include, for example, bottles, vials, bags and syringes. The container may be formed from a variety of materials such as glass, plastic (such as polyvinyl chloride or polyolefin), or metal alloy (such as stainless steel or hastelloy). An exemplary container is a 300 cc metal alloy container (e.g., for storing at −20° C.). Another exemplary container may be 10-50 cc glass vial (e.g., for storing at 2-8° C.). For example, the container may be 10 cc, 15 cc, 20 cc, or 50 cc glass vials. The container holds the formulation and the label on, or associated with, the container may indicate directions for use. The article of manufacture may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: Drug Substance (DS) Formulation Stability

For DS studies, formulations were filled into stainless steel mini-cans and placed at the appropriate storage conditions to evaluate atezolizumab frozen storage and accelerate stability. To formulate the DS for the studies, ultrafiltration diafiltration pools of atezolizumab were buffer exchanged into appropriate buffer systems (e.g. histidine acetate, histidine hydrochloride, histidine acetate with arginine) and then polysorbate 20, and methionine were added to the ultrafiltration diafiltration material to formulate the DS.

### DS Stability After Multiple Freeze-Thaw Cycles

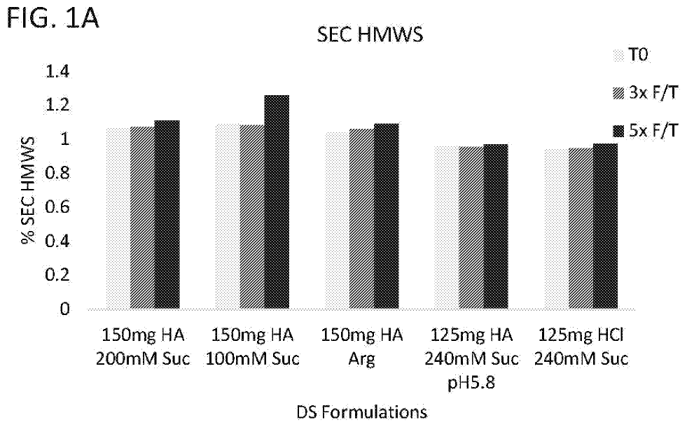

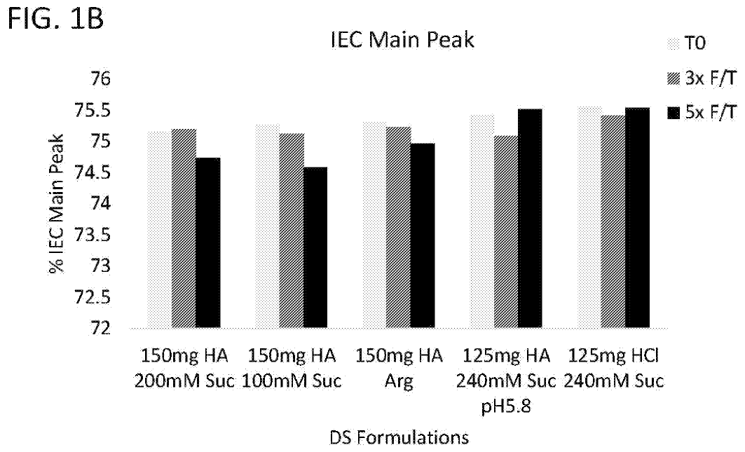

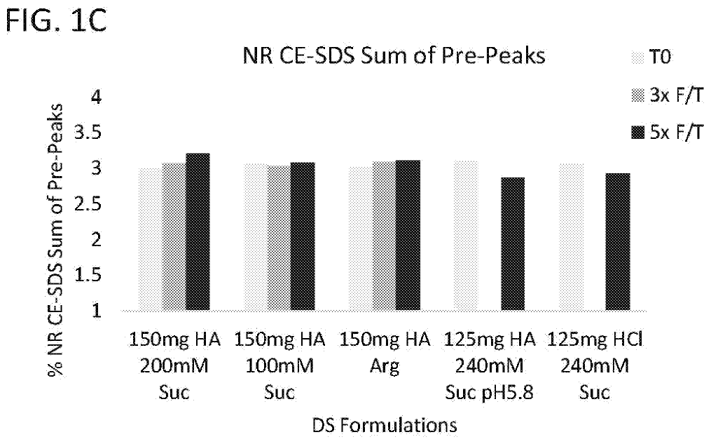

FIG. 1A-1C shows the levels of high molecular weight species (HMWS) (FIG. 1A), ion exchange chromatography (IEC) main peak percentages (FIG. 1B), and non-reducing capillary electrophoresis-SDS (NR CE-SDS) pre-peak sums (FIG. 1C) of various DS formulations after multiple freeze/thaw cycles. All formulations comprised 150 mg/ml or 125 mg/ml of atezolizumab (indicated as 150 mg or 125 mg in the figure), 20 mM histidine acetate (HA) or histidine hydrochloride (HCl), 10 mM methionine, and 0.06% (w/v) polysorbate 20. Unless stated otherwise, all formulations were at pH 5.5. Formulation containing low sucrose concentration (e.g. 100 mM) was insufficient to maintain stability through multiple freeze thaw cycles at high protein concentrations. As a result of these experiments, a sucrose concentration of 240 mM for formulations comprised of histidine acetate or histidine hydrochloride was selected to support 5 freeze/thaw (F/T) cycles.

### DS Stability at 25° C.

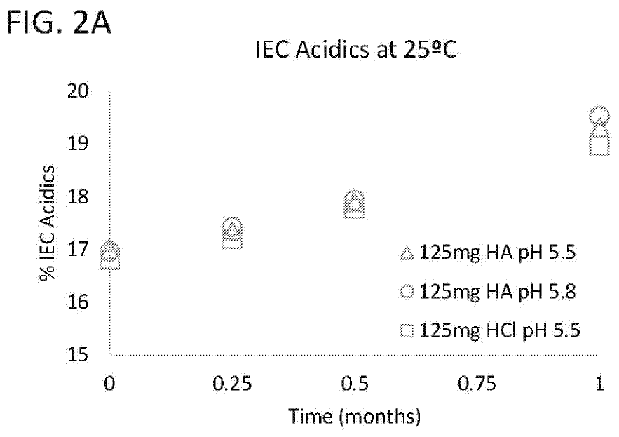

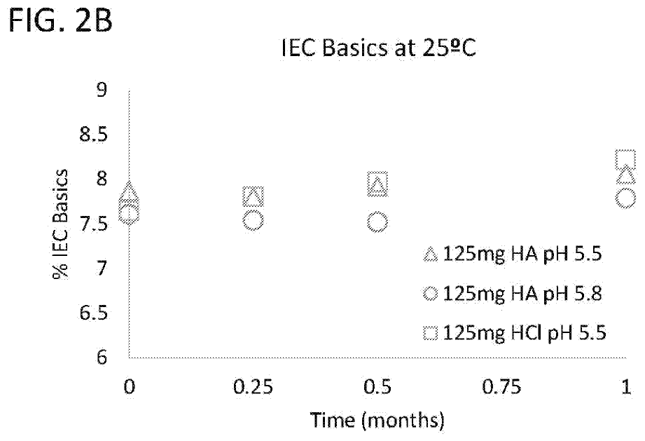

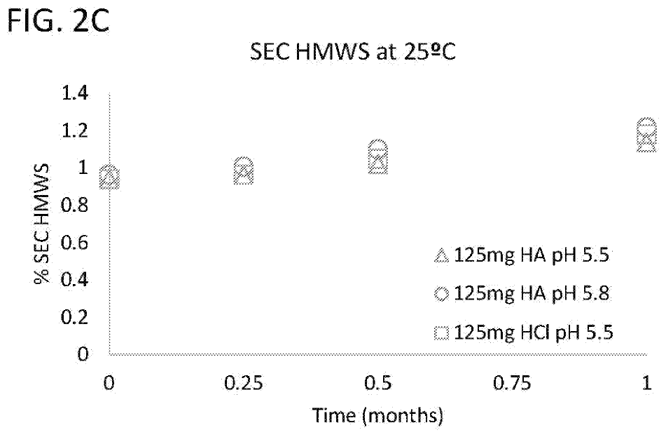

FIG. 2A-2C shows the levels of acidic species (FIG. 2A), basic species (FIG. 2B) and HMWS (FIG. 2C) of various DS formulations after up to 1 month at 25° C. All formulations comprised 125 mg/ml of atezolizumab (indicated as 125 mg in the figure), 20 mM histidine acetate (HA) or histidine hydrochloride (HCl), 10 mM methionine, and 0.06% (w/v) polysorbate 20. IEC showed a lower percentage of acidics and higher percentage of basics in with the pH 5.5 formulations.

### Example 2: Drug Product (DP) Formulation Stability

For the DP stability studies, the formulations were filled into glass vials and placed at the appropriate storage conditions to evaluate stability of atezolizumab in different buffer systems and excipients. To formulate, ultrafiltration diafiltration pools of atezolizumab were buffer exchange into appropriate buffer systems (e.g. histidine acetate, histidine hydrochloride, histidine acetate with arginine) and then polysorbate 20, methionine and recombinant human hyaluronidase were added to the ultrafiltration diafiltration material to formulate the DPs.

### DP Stability at 25° C.

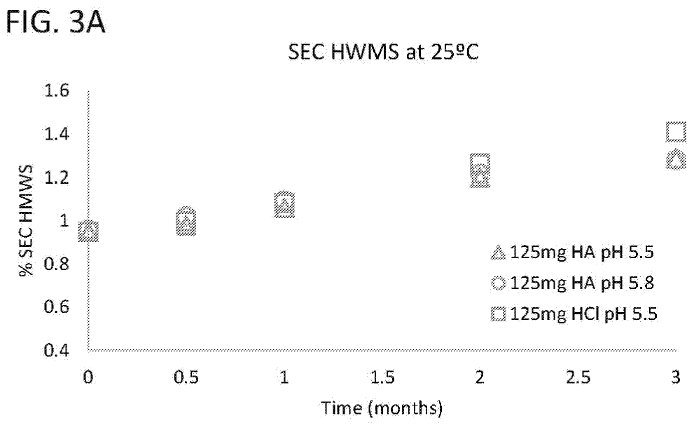

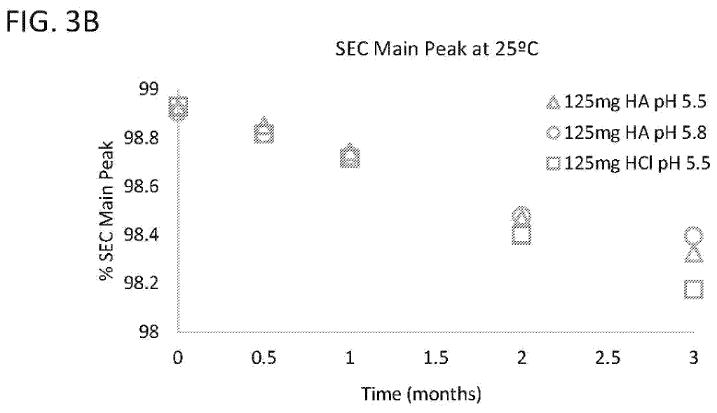

FIG. 3A-3B shows the levels of HMWS (FIG. 3A) and SEC main peak percentages (FIG. 3B) of DP formulations after up to 3 months at 25° C. All formulations comprised 125 mg/ml atezolizumab (indicated as 125 mg in the figure), 20 mM histidine acetate or histidine hydrochloride, 240 mM sucrose, 10 mM methionine, 0.06% polysorbate 20, and 2000 U/ml recombinant human hyaluronidase (rHuPH20). The formulation comprising histidine acetate at pH 5.8 had a higher SEC main peak percentage compared to the other two formulations (FIG. 3B). The formulation comprising histidine hydrochloride at pH 5.5 had a higher percentage of HMWS compared to the other two formulations (FIG. 3A). Overall, the histidine acetate formulations showed slightly slower degradation by SEC when compared with histidine hydrochloride formulation.

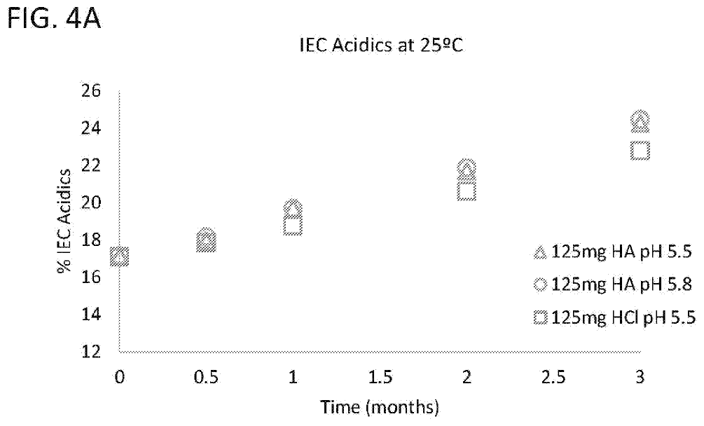

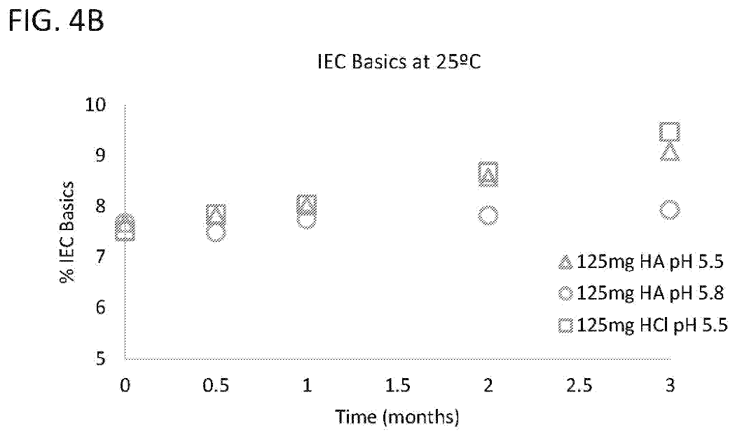

FIG. 4A-4B shows the levels of acidic species (FIG. 4A) and basic species (FIG. 4B) in DP formulations after up to 3 months at 25° C. All formulations comprised 125 mg/ml atezolizumab (indicated as 125 mg in the figure), 20 mM histidine acetate or histidine hydrochloride, 240 mM sucrose, 10 mM methionine, 0.06% polysorbate 20, and 2000 U/ml recombinant human hyaluronidase (rHuPH20). Overall, the IEC main peak degradation rates between all three formulations were similar. However, the formation of acidic and basic species was different between the 3 formulations. The formulation comprising histidine acetate at pH 5.8 had a lower percentage of basic species compared to the other two formulations (FIG. 4B). The formulations comprising histidine acetate had higher percentages of acidic species compared to the formulation comprising histidine hydrochloride (FIG. 4A).

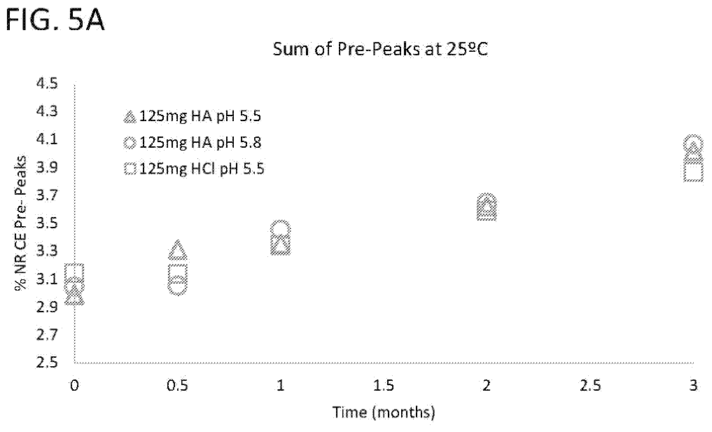

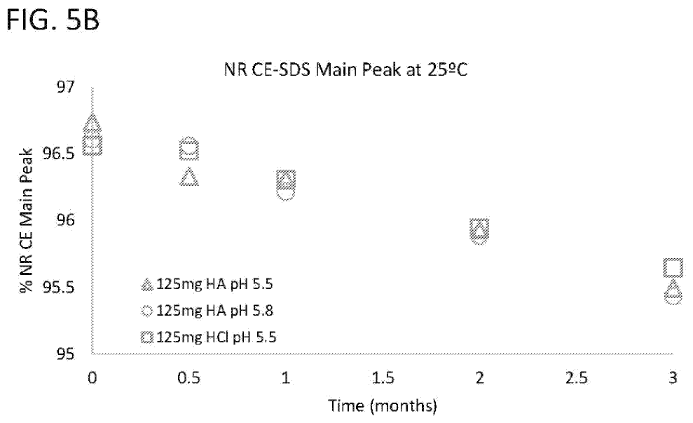

FIG. 5A-5B shows the percentages of pre-peaks (FIG. 5A) and NR CE-SDS main peak (FIG. 5B) in DP formulations after up to 3 months at 25° C. All formulations comprised 125 mg/ml atezolizumab (indicated as 125 mg in the figure), 20 mM histidine acetate or histidine hydrochloride, 240 mM sucrose, 10 mM methionine, 0.06% polysorbate 20, and 2000 U/ml recombinant human hyaluronidase (rHuPH20).

### DP Stability at 40° C.

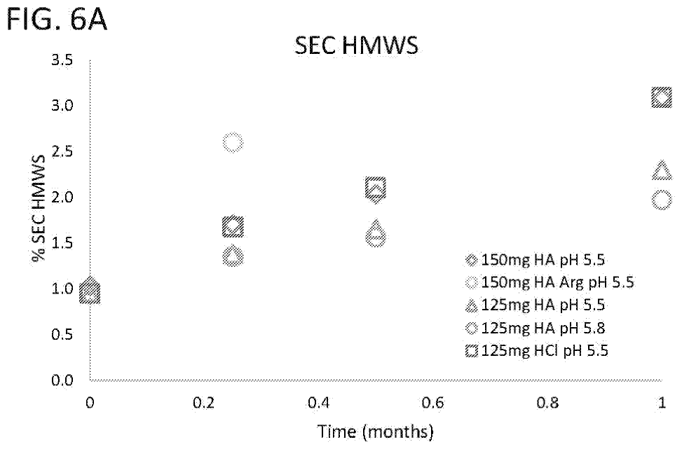

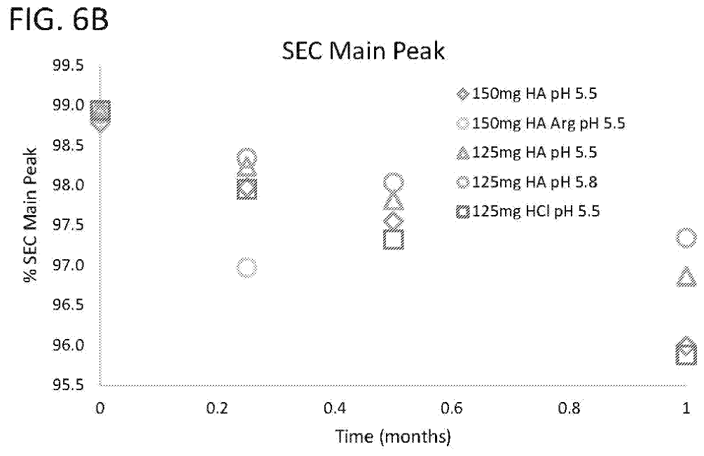

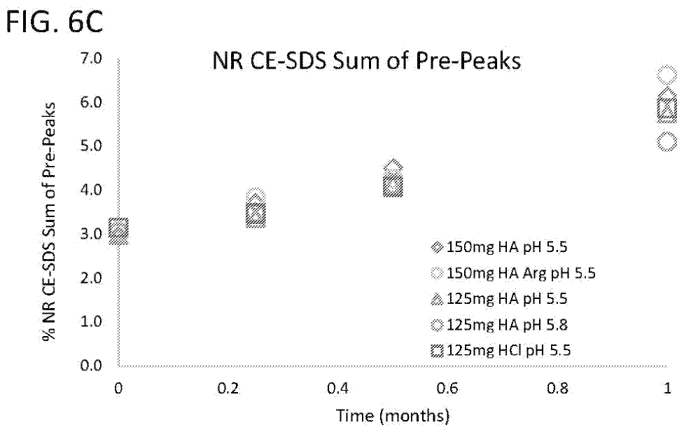

FIG. 6A-6C shows the levels of HMWS (FIG. 6A), SEC main peak percentages (FIG. 6B), and NR CE-SDS sum of pre-peaks (FIG. 6C) in DP formulations after up to 1 month at 40° C. All formulations comprised 150 mg/ml or 125 mg/ml of atezolizumab (indicated as 150 mg or 125 mg in the figure), 200-240 mM sucrose, 10 mM methionine, 0.06% polysorbate 20, and 2000 U/ml recombinant human hyaluronidase (rHuPH20). The formulations comprising 125 mg/ml atezolizumab and histidine acetate had less HMWS (FIG. 6A), a higher main peak percentage (FIG. 6B), and lower sum of NR-CE SDS pre-peaks (FIG. 6C) than the other formulations. With increasing protein concentrations, the HMWS also increased. In terms of pH, higher pH (e.g. 5.8) decreased HMWS formation and fragmentation. The addition of arginine also contributed to an increase in HMWS. Although arginine may increase solubility however it failed to maintain physical stability (e.g. increase of HMWS) of atezolizumab.

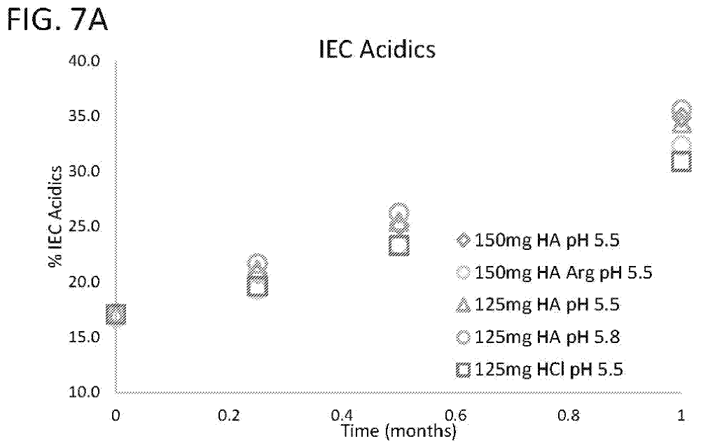

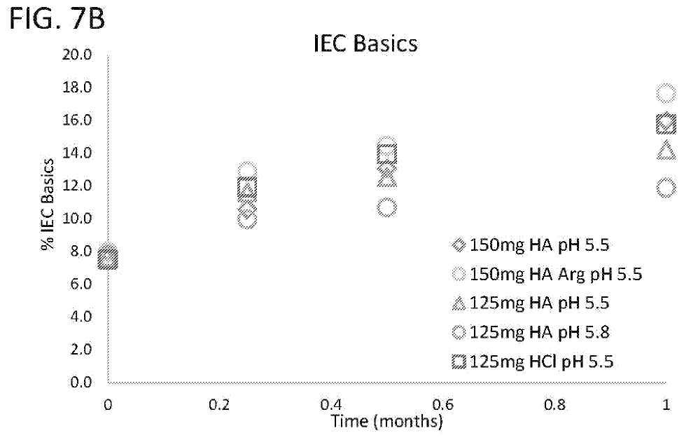

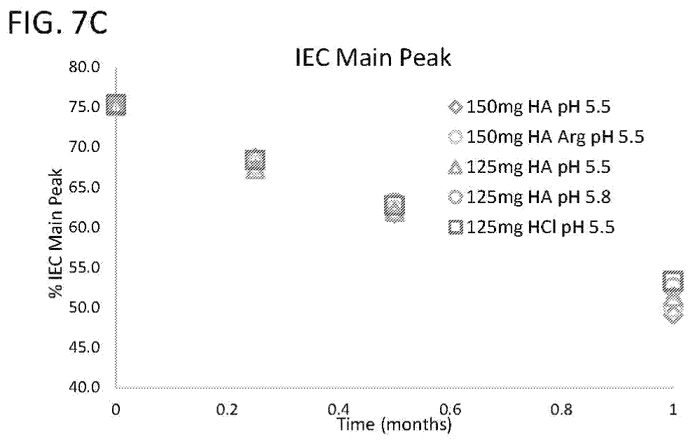

FIG. 7A-7C shows the levels of the levels of acidic species (FIG. 7A), basic species (FIG. 7B), and percentage of IEC main peak (FIG. 7C) in DP formulations after up to 1 month at 40° C. All formulations comprised 150 mg/ml or 125 mg/ml of atezolizumab (indicated as 150 mg or 125 mg in the figure), 200-240 mM sucrose, 10 mM methionine, 0.06% polysorbate 20, and 2000 U/ml recombinant human hyaluronidase (rHuPH20). Formulations with histidine acetate buffer had higher levels of acidic species compared to formulations with histidine hydrochloride buffer or histidine acetate+arginine buffer (FIG. 7A). The formulation comprising 125 mg/ml atezolizumab and histidine acetate at pH 5.8 had lower levels of basic species compared to the other formulations (FIG. 7B).

Based on the product stability results presented, the formulation comprising of 125 mg/mL atezolizumab and histidine acetate at pH 5.8 was selected for the formulation for atezolizumab.

### Example 3: Polysorbate 20 Stability

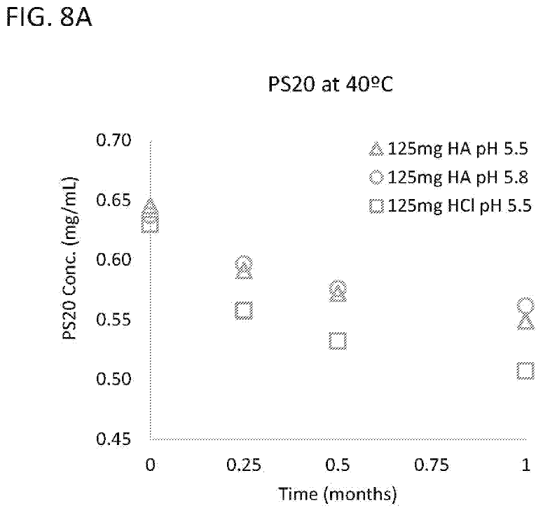

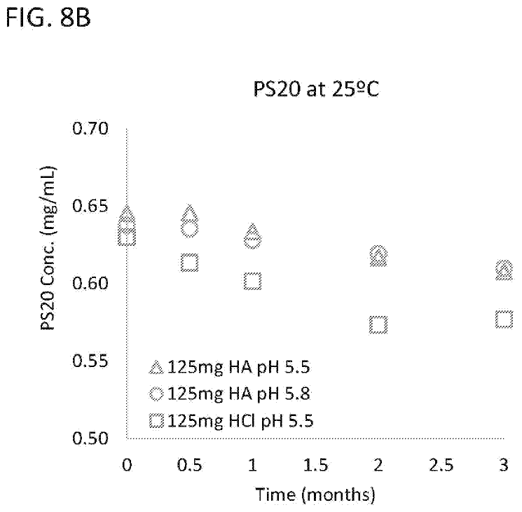

FIG. 8A-8B shows the stability of polysorbate 20 at 40° C. (FIG. 8A) and at 25° C. (FIG. 8B) for up to 3 months in various DP formulations. All formulations comprised 125 mg/ml atezolizumab (indicated as 125 mg in the figure), 20 mM histidine acetate or histidine hydrochloride, 240 mM sucrose, 10 mM methionine, 0.06% polysorbate 20, and 2000 U/ml recombinant human hyaluronidase (rHuPH20). Polysorbate 20 showed less degradation in the formulation comprising histidine acetate and pH 5.8 at both 40° C. and 25° C. after 3 months compared to the other two formulations.

The data from 25° C. experiment described above was used to calculate the theoretical amount of polysorbate 20 lost after 6 months at 25° C. As can be seen in the table below, polysorbate 20 is predicted to show less degradation after 6 months in the formulation comprising histidine acetate and pH 5.8 compared to the other two formulations.

Theoretical PS20 Loss After 6 Months at 25° C.

| 125 mg atezolizumab | 125 mg atezolizumab | 125 mg atezolizumab |
|---|---|---|
| Histidine acetate | Histidine acetate | Histidine hydrochloride |
| pH 5.5 | pH 5.8 | pH 5.5 |
| 15% | 9% | 18% |

In addition to product stability of atezolizumab, the formulation comprised of histidine acetate and pH 5.8 most effectively maintained polysorbate 20 stability.

### Example 4: rHuPH20 Activity

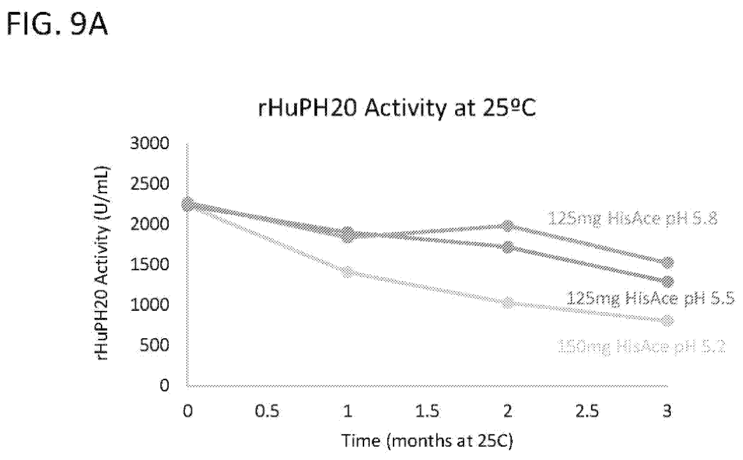

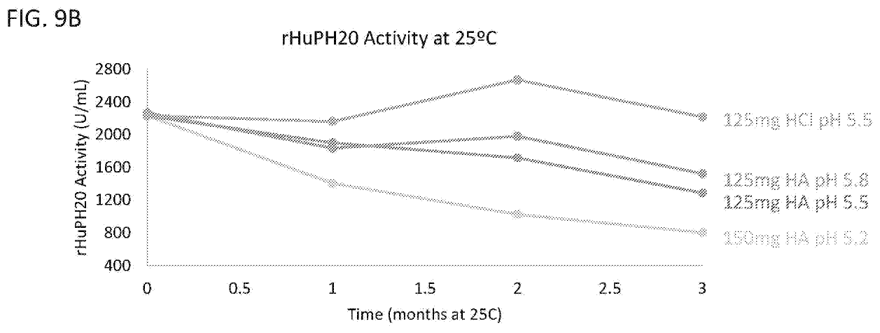

FIG. 9A-9B shows rHuPH20 activity assays with various DP formulations at 25° C. for up to 3 months. All formulations comprised 150 mg/ml or 125 mg/ml of atezolizumab (indicated as 150 mg or 125 mg in the figure), 20 mM histidine acetate or histidine hydrochloride, 240 mM sucrose, 10 mM methionine, 0.06% polysorbate 20, and 2000 U/ml recombinant human hyaluronidase (rHuPH20). Formulations comprising histidine acetate at pH 5.8 maintained rHuPH20 activity at higher levels than formulations comprising histidine acetate at pH 5.5. Increasing the pH also increased rHuPH20 stability at 25 C. Although the formulation comprised of histidine hydrochloride provided better stability of rHuPH20 at accelerated conditions (FIG. 9B) when compared to other formulations, histidine hydrochloride was not well suited for atezolizumab. Although a slight decrease in rHuPH20 activity was observed at accelerated conditions for formulations comprised of histidine acetate, this was not observed at 5° C. storage. As a result, the formulation comprised of histidine acetate and pH 5.8 was selected for atezolizumab.

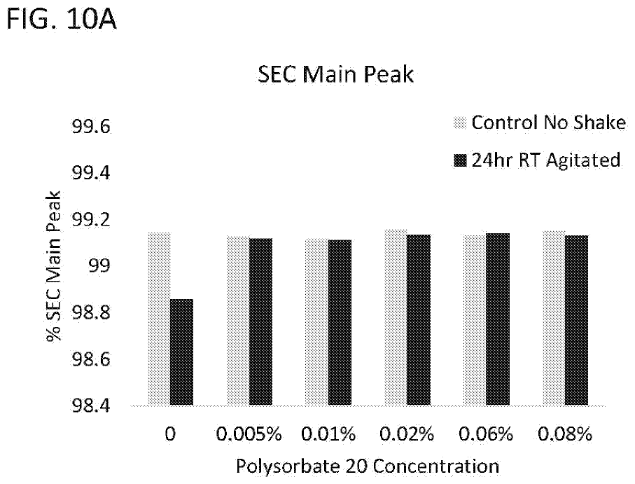

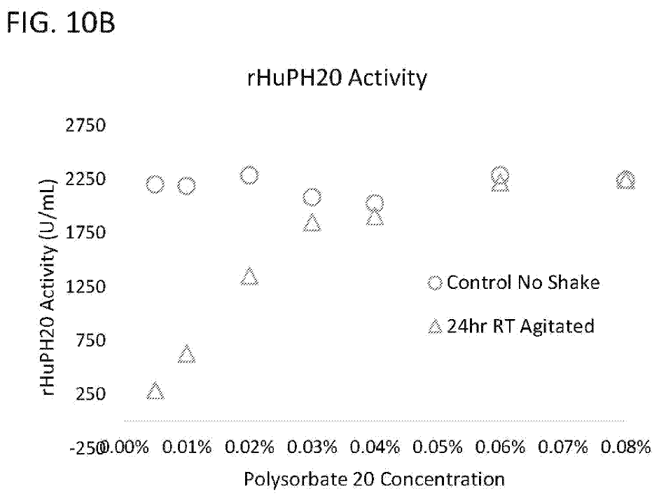

FIG. 10A-10B shows rHuPH20 activity in formulations comprising different concentrations of polysorbate in the while being agitate for 24 hours. Higher concentrations of polysorbate maintained rHuPH20 activity at higher levels under agitation at room temperature. A minimum of 0.03% (w/v) polysorbate 20 is required prevent rHuPH20 loss against agitation. With the consideration of polysorbate 20 release criteria and possible polysorbate degradation over shelf-life, a polysorbate 20 level of 0.06% (w/v) was selected for the formulation.

### Example 5: Drug Product (DP) Formulation Viscosity

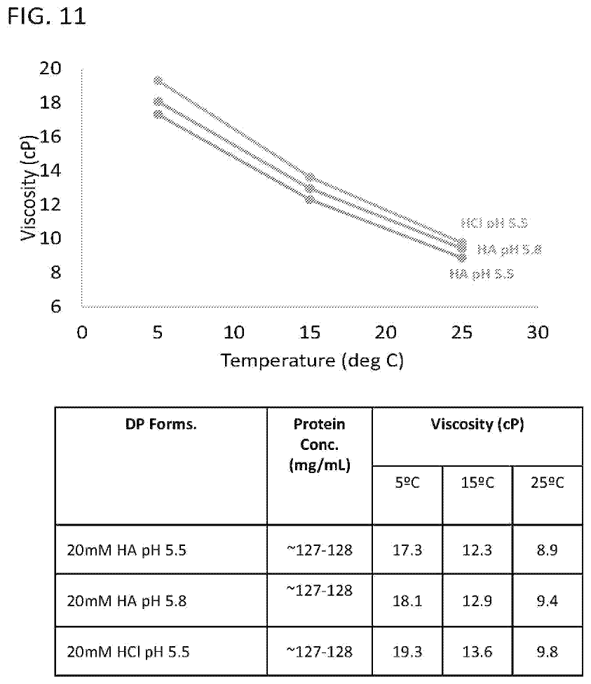

FIG. 11 shows the viscosities of various DP formulations at temperatures between 5° C. and 25° C. All formulations comprised 127-128 mg/ml atezolizumab, 20 mM histidine acetate or histidine hydrochloride, 240 mM sucrose, 10 mM methionine, 0.06% polysorbate 20, and 2000 U/ml recombinant human hyaluronidase (rHuPH20). The formulation comprising histidine hydrochloride had the highest viscosity at all temperatures evaluated.

Based on the formulation screening described in these examples, a DP formulation comprising 125 mg/ml atezolizumab, 20 mM histidine acetate, 240 mM sucrose, 10 mM methionine, 0.06% polysorbate 20, 2000 U/ml recombinant human hyaluronidase (rHuPH20), at pH 5.8 was selected for the atezolizumab formulation for subcutaneous administration.

What is claimed is:

1. A liquid pharmaceutical formulation, the formulation comprising a monoclonal anti-PD-L1 antibody in a concentration of about 100 g/L to about 150 g/L, histidine acetate in a concentration of about 15 mM to about 25 mM, sucrose in a concentration of about 200 mM to about 280 mM, polysorbate in a concentration of about 0.04% (w/v) to about 0.08% (w/v), methionine in a concentration of about 5 mM to about 15 mM, and pH of about 5.6 to about 6.0, wherein the monoclonal antibody comprises
(a) a light chain variable region comprising:
(1) HVR-L1 comprising the amino acid sequence RASQDVSTAVA (SEQ ID NO: 1);
(2) HVR-L2 comprising the amino acid sequence SASFLYS (SEQ ID NO:2);
(3) HVR-L3 comprising the amino acid sequence QQYLYHPAT (SEQ ID NO:3); and
(b) a heavy chain variable region comprising:
(1) HVR-H1 comprising the amino acid sequence GFTFSDSWIH (SEQ ID NO:4);
(2) HVR-H2 comprising the amino acid sequence AWISPYGGSTYYADSVKG (SEQ ID NO:5);
(3) HVR-H3 comprising the amino acid sequence WPGGFDY (SEQ ID NO:6).

2. The liquid pharmaceutical formulation of claim 1, wherein the monoclonal antibody in the formulation is in a concentration of about 120 g/L to about 130 g/L.

3. The liquid pharmaceutical formulation of claim 1, wherein the monoclonal antibody in the formulation is in a concentration of about 125 g/L.

4. The liquid pharmaceutical formulation of any one of claims 1-3, wherein the histidine acetate is in a concentration of about 17 mM to about 22 mM.

5. The liquid pharmaceutical formulation of any one of claims 1-3, wherein the histidine acetate is in a concentration of about 20 mM.

6. The liquid pharmaceutical formulation of any one of claims 1-5, wherein the sucrose is in a concentration of about 220 mM to about 260 mM.

7. The liquid pharmaceutical formulation of any one of claims 1-5, wherein the sucrose is in a concentration of about 240 mM.

8. The liquid pharmaceutical formulation of any one of claims 1-7, wherein the pH is about 5.8.

9. The liquid pharmaceutical formulation of any one of claims 1-8, wherein the polysorbate in the formulation is polysorbate 20.

10. The liquid pharmaceutical formulation of any one of claims 1-9, wherein the polysorbate is in a concentration of about 0.05% (w/v) to about 0.07% (w/v).

11. The liquid pharmaceutical formulation of any one of claims 1-9, wherein the polysorbate is in a concentration of about 0.06% (w/v).

12. The liquid pharmaceutical formulation of any one of claims 1-11, wherein the methionine is in a concentration of about 10 mM.

13. The liquid pharmaceutical formulation of any one of claims 1-12, wherein the formulation further comprises a hyaluronidase enzyme.

14. The liquid pharmaceutical formulation of claim 13, wherein the hyaluronidase enzyme is recombinant human hyaluronidase (rHuPH20).

15. The liquid pharmaceutical formulation of claim 13 or 14, wherein the hyaluronidase enzyme is in a concentration of about 1000 U/ml to about 3000 U/ml.

16. The liquid pharmaceutical formulation of claim 13 or 14, wherein the hyaluronidase enzyme is in a concentration of about 2000 U/ml.

17. A liquid pharmaceutical formulation, the formulation comprising a monoclonal anti-PD-L1 antibody in a concentration of about 100 g/L to about 150 g/L, histidine acetate in a concentration of about 15 mM to about 25 mM, sucrose in a concentration of about 200 mM to about 280 mM, polysorbate in a concentration of about 0.01% (w/v) to about 0.03% (w/v), and pH of about 5.3 to about 5.7, wherein the monoclonal antibody comprises
(a) a light chain variable region comprising:
(1) HVR-L1 comprising the amino acid sequence RASQDVSTAVA (SEQ ID NO: 1);
(2) HVR-L2 comprising the amino acid sequence SASFLYS (SEQ ID NO:2);
(3) HVR-L3 comprising the amino acid sequence QQYLYHPAT (SEQ ID NO:3); and
(b) a heavy chain variable region comprising:
(1) HVR-H1 comprising the amino acid sequence GFTFSDSWIH (SEQ ID NO:4);
(2) HVR-H2 comprising the amino acid sequence AWISPYGGSTYYADSVKG (SEQ ID NO:5);
(3) HVR-H3 comprising the amino acid sequence WPGGFDY (SEQ ID NO:6).

18. The liquid pharmaceutical formulation of claim 17, wherein the monoclonal antibody in the formulation is in a concentration of about 120 g/L to about 130 g/L.

19. The liquid pharmaceutical formulation of claim 17, wherein the monoclonal antibody in the formulation is in a concentration of about 125 g/L.

20. The liquid pharmaceutical formulation of any one of claims 17-19, wherein the histidine acetate is in a concentration of about 17 mM to about 22 mM.

21. The liquid pharmaceutical formulation of any one of claims 17-19, wherein the histidine acetate is in a concentration of about 20 mM.

22. The liquid pharmaceutical formulation of any one of claims 17-21, wherein the sucrose is in a concentration of about 220 mM to about 260 mM.

23. The liquid pharmaceutical formulation of any one of claims 17-21, wherein the sucrose is in a concentration of about 240 mM.

24. The liquid pharmaceutical formulation of any one of claims 17-23, wherein the pH is about 5.5.

25. The liquid pharmaceutical formulation of any one of claims 17-24, wherein the polysorbate in the formulation is polysorbate 20.

26. The liquid pharmaceutical formulation of any one of claims 17-25, wherein the polysorbate is in a concentration of about 0.02% (w/v).

27. The liquid pharmaceutical formulation of any one of claims 17-26, wherein the formulation is mixed with a hyaluronidase enzyme prior to being administered to a subject.

28. The liquid pharmaceutical formulation of claim 27, wherein the hyaluronidase enzyme is recombinant human hyaluronidase (rHuPH20).

29. The liquid pharmaceutical formulation of claim 27 or 28, wherein the hyaluronidase enzyme concentration in the mixture is about 1000 U/ml to about 3000 U/ml.

30. The liquid pharmaceutical formulation of claim 27 or 28, wherein the hyaluronidase enzyme concentration in the mixture is about 2000 U/ml.

31. The liquid pharmaceutical formulation of any one of claims 1-30, wherein the monoclonal antibody is not subject to prior lyophilization.

32. The liquid pharmaceutical formulation of any one of claims 1-31, wherein the monoclonal antibody is a humanized antibody.

33. The liquid pharmaceutical formulation of any one of claims 1-32, wherein the monoclonal antibody comprises a light chain variable region comprising the amino acid sequence of SEQ ID NO:7, and a heavy chain variable region comprising the amino acid sequence of SEQ ID NO:8.

34. The liquid pharmaceutical formulation of any one of claims 1-33, wherein the monoclonal antibody is a full length antibody.

35. The liquid pharmaceutical formulation of claim 34, wherein the monoclonal antibody is an IgG1 antibody.

36. The liquid pharmaceutical formulation of any one of claims 1-35, wherein the monoclonal antibody comprises a light chain comprising the amino acid sequence of SEQ ID NO:9, and a heavy comprising the amino acid sequence of SEQ ID NO: 10.

37. The liquid pharmaceutical formulation of any one of claims 1-36, wherein the monoclonal antibody is stored in a glass vial or a metal alloy container.

38. The liquid pharmaceutical formulation of claim 37, wherein the metal alloy is 316L stainless steel or hastelloy.

39. The liquid pharmaceutical formulation of any one of claims 1-38, wherein the formulation is stable at 2-8° C. for at least 6 months.

40. The liquid pharmaceutical formulation of any one of claims 1-38, wherein the formulation is stable at 2-8° C. for at least 12 months.

41. The liquid pharmaceutical formulation of any one of claims 1-38, wherein the formulation is stable at 2-8° C. for at least 24 months.

42. The liquid pharmaceutical formulation of any one of claims 39-41, wherein the antibody in the formulation retains at least about 80% of its biological activity after storage.

43. The liquid pharmaceutical formulation of claim 42, wherein the biological activity is measured by antibody binding to PD-L1.

44. The liquid pharmaceutical formulation of any one of claims 1-43, wherein the formulation is sterile.

45. The liquid pharmaceutical formulation of any one of claims 1-44, wherein the formulation is suitable to be administered to a subject.

46. The liquid pharmaceutical formulation of any one of claims 1-45, wherein the formulation is for subcutaneous administration.

47. An article of manufacture comprising a container holding the liquid pharmaceutical formulation of any one of claims 1-46.

48. The article of manufacture of claim 47, wherein the container is a glass vial or a metal alloy container.

49. The article of manufacture of claim 48, wherein the metal alloy is 316L stainless steel or hastelloy.

50. A kit comprising a container holding the liquid pharmaceutical formulation of any one of claims 1-46.

51. A method of treating a disease or disorder in a subject comprising administering an effective amount of the liquid pharmaceutical formulation of any one of claims 1-46 to the subject, wherein the disease or disorder is selected from the group consisting of infection, cancer, and inflammatory disease.

52. The method of claim 51, wherein the disease or disorder is cancer.

53. The method of claim 52, wherein the cancer is selected from the group consisting of non-small cell lung cancer, small cell lung cancer, urothelial carcinoma, and breast cancer.

54. The method of claim 53, wherein the breast cancer is triple negative breast cancer.

55. The method of any one of claims 51-54, wherein the subject is a human.


SEQUENCE LISTING (10 sequences)

SEQ ID NO: 1 - protein, 11 aa, Artificial Sequence
  Synthetic Construct
RASQDVSTAVA

SEQ ID NO: 2 - protein, 7 aa, Artificial Sequence
  Synthetic Construct
SASFLYS

SEQ ID NO: 3 - protein, 9 aa, Artificial Sequence
  Synthetic Construct
QQYLYHPAT

SEQ ID NO: 4 - protein, 10 aa, Artificial Sequence
  Synthetic Construct
GFTFSDSWIH

SEQ ID NO: 5 - protein, 18 aa, Artificial Sequence
  Synthetic Construct
AWISPYGGSTYYADSVKG

SEQ ID NO: 6 - protein, 7 aa, Artificial Sequence
  Synthetic Construct
WPGGFDY

SEQ ID NO: 7 - protein, 107 aa, Artificial Sequence [sequence omitted]

SEQ ID NO: 8 - protein, 118 aa, Artificial Sequence [sequence omitted]

SEQ ID NO: 9 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 447 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG